(19) United States
(12) Patent Application Publication
(10) Pub. No.: US 2017/0219705 A1
(43) Pub. Date: Aug. 3, 2017

(54) ULTRASONIC DIAGNOSIS APPARATUS AND STORAGE MEDIUM

(71) Applicant: Toshiba Medical Systems Corporation, Otawara-shi (JP)

(72) Inventors: Yukifumi Kobayashi, Utsunomiya (JP); Masaru Ogasawara, Musashimurayama (JP); Shinichi Hashimoto, Otawara (JP); Tomokazu Fujii, Nasushiobara (JP); Shunsuke Satoh, Nasushiobara (JP)

(73) Assignee: Toshiba Medical Systems Corporation, Otawara-shi (JP)

(21) Appl. No.: 15/421,995

(22) Filed: Feb. 1, 2017

(30) Foreign Application Priority Data
Feb. 3, 2016 (JP) 2016-019225
Nov. 15, 2016 (JP) 2016-222377

(51) Int. Cl. G01S 15/89; A61B 8/13; A61B 8/08; G01S 7/52; A61B 8/00

(52) U.S. Cl. CPC G01S 15/8995; G01S 7/52055; G01S 15/8993; A61B 8/4254; A61B 8/463; A61B 8/5238; A61B 8/54; A61B 8/13; G01S 15/8934; G01S 15/8977; A61B 2090/365

(57) ABSTRACT

According to one embodiment, an ultrasonic diagnosis apparatus includes processing circuitry and a display. The processing circuitry executes a load process of loading predetermined data from volume data stored in other apparatus. The processing circuitry executes a reconstruction process of reconstructing volume data from the loaded data. The processing circuitry executes a registration process in such a manner as to register the positions of the displayed ultrasonic image and slice image based on the loaded data. The processing circuitry executes a control process of controlling, after the registration process, the display in such a manner as to interlock-display the ultrasonic image and slice image.

### CROSS-REFERENCE TO RELATED APPLICATIONS

This application is based upon and claims the benefit of priority from prior Japanese Patent Applications No. 2016-019225, filed on Feb. 3, 2016, and No. 2016-222377, filed on Nov. 15, 2016, the entire contents of all of which are incorporated herein by reference.

### FIELD

Embodiments described herein relate generally to an ultrasonic diagnosis apparatus and a storage medium.

### BACKGROUND

In recent years, in ultrasonic diagnosis apparatuses, a fusion function, which executes guide display for an ultrasonic image, has begun to be used, for example, at a time of puncture or radio-frequency ablation (RFA) treatment. The fusion function is a function of registering an ultrasonic image, which is acquired in real time, with a slice image based on pre-acquired volume data, and interlock-displaying the ultrasonic image and the slice image.

In this kind of fusion function, the position and angle of an ultrasonic probe, which correspond to a currently displayed ultrasonic image, are acquired by using a transmitter which is disposed on or near the main body of an ultrasonic diagnosis apparatus, and a sensor (receiver) which is attached to the ultrasonic probe.

Subsequently, in the fusion function, for example, volume data (DICOM (Digital Imaging and Communications in Medicine) data), which was acquired by other modality (medical diagnosis apparatus) such as an X-ray computed tomography (hereinafter referred to as “CT”) apparatus or a magnetic resonance imaging (hereinafter “MRI”) apparatus, is loaded in the ultrasonic diagnosis apparatus. In the fusion function, if a load time has passed, an MPR (Multi-Planar Reconstruction) image (slice image) is displayed based on the volume data.

Thereafter, in the fusion function, an identical cross section is displayed by searching a common target between the ultrasonic image and the MPR image, and the position/angle information relating to an identical position of the identical cross section is registered. To register the position/angle information is also called “registration”.

Then, in the fusion function, with the movement of the ultrasonic probe, the same cross section as a varying ultrasonic image can be displayed by the MPR image. In this fusion function, at a time of puncture or RFA treatment, a tumor or the like, which is difficult to ascertain by the ultrasonic image, can be guide-displayed by the MPR image.

The above-described fusion function has no problem in usual cases. However, according to the study by the inventor, there is room for improvement with respect to the following point.

For example, in the fusion function, unless all volume data is loaded, a volume cannot be constructed, and it is not possible to advance to a subsequent step such as a target search or registration. Thus, if the load time is long, a workflow would be hindered. Here, the load time of volume data has such a characteristic that the load time becomes longer in proportion to the data amount. In addition, with the enhancement in resolution of the medical diagnosis apparatus in recent years, there is a tendency that the number of slices of CT data, for instance, increases, and the data amount increases. It is thus estimated that the load time of volume data will increase in the future. Therefore, the fusion function has room for improvement in that the workflow is hindered due to the load time of volume data.

The object is to provide an ultrasonic diagnosis apparatus and a storage medium, which can reduce a hindrance to a workflow due to a load time of volume data.

### BRIEF DESCRIPTION OF THE DRAWINGS

FIG. 1 is a schematic view illustrating an ultrasonic diagnosis apparatus according to a first embodiment, and a peripheral configuration thereof.

FIG. 2 is a schematic view for describing a position sensor and a transmitter in the embodiment.

FIG. 3 is a schematic view for describing a first load method in the embodiment.

FIG. 4 is a schematic view for describing a second load method in the embodiment.

FIG. 5 is a schematic view for describing a third load method in the embodiment.

FIG. 6 is a schematic view for describing a fourth load method in the embodiment.

FIG. 7 is a schematic view for describing a variation of a registration process in the embodiment.

FIG. 8 is a schematic view for describing a load process, etc. in the embodiment.

FIG. 9 is a schematic view for describing the load process, etc. in the embodiment.

FIG. 10 is a flowchart for describing an operation in the embodiment.

FIG. 11 is a schematic view for describing the operation in the embodiment.

FIG. 12 is a schematic view illustrating an ultrasonic diagnosis apparatus according to a second embodiment, and a peripheral configuration thereof.

FIG. 13 is a schematic view for describing an operation in the embodiment.

FIG. 14 is a flowchart for describing the operation in the embodiment.

FIG. 15 is a schematic view for schematically describing the configuration of an ultrasonic diagnosis apparatus according to a third embodiment.

FIG. 16 is a flowchart for describing an operation in the embodiment.

FIG. 17 is a schematic view for schematically describing the configuration of an ultrasonic diagnosis apparatus according to a fourth embodiment.

FIG. 18 is a flowchart for describing an operation in the embodiment.

FIG. 19 is a schematic view for schematically describing the configuration of an ultrasonic diagnosis apparatus according to a fifth embodiment.

FIG. 20 is a flowchart for describing an operation in the embodiment.

FIG. 21 is a schematic view illustrating an ultrasonic diagnosis apparatus according to a sixth embodiment, and a peripheral configuration thereof.

FIG. 22 is a schematic view for describing the outline of the ultrasonic diagnosis apparatus and a server apparatus in the embodiment.

FIG. 23 is a flowchart for describing an operation in the embodiment.

### DETAILED DESCRIPTION

In general, according to one embodiment, an ultrasonic diagnosis apparatus includes position acquisition circuitry, processing circuitry and a display.

The position acquisition circuitry acquires position information of an ultrasonic probe.

The processing circuitry executes a load process of loading predetermined data from volume data stored in other apparatus.

The processing circuitry executes a reconstruction process of reconstructing volume data from the loaded data.

The display displays in parallel a slice image of the reconstructed volume data and an ultrasonic image based on an output of the ultrasonic probe.

The processing circuitry executes a registration process in such a manner as to register the positions of the displayed ultrasonic image and slice image based on the loaded data.

The processing circuitry executes a control process of controlling, after the registration process, the display in such a manner as to interlock-display the ultrasonic image and slice image in accordance with the position information.

Hereinafter, ultrasonic diagnosis apparatuses, etc. according to embodiments will be described with reference to the accompanying drawings

### First Embodiment

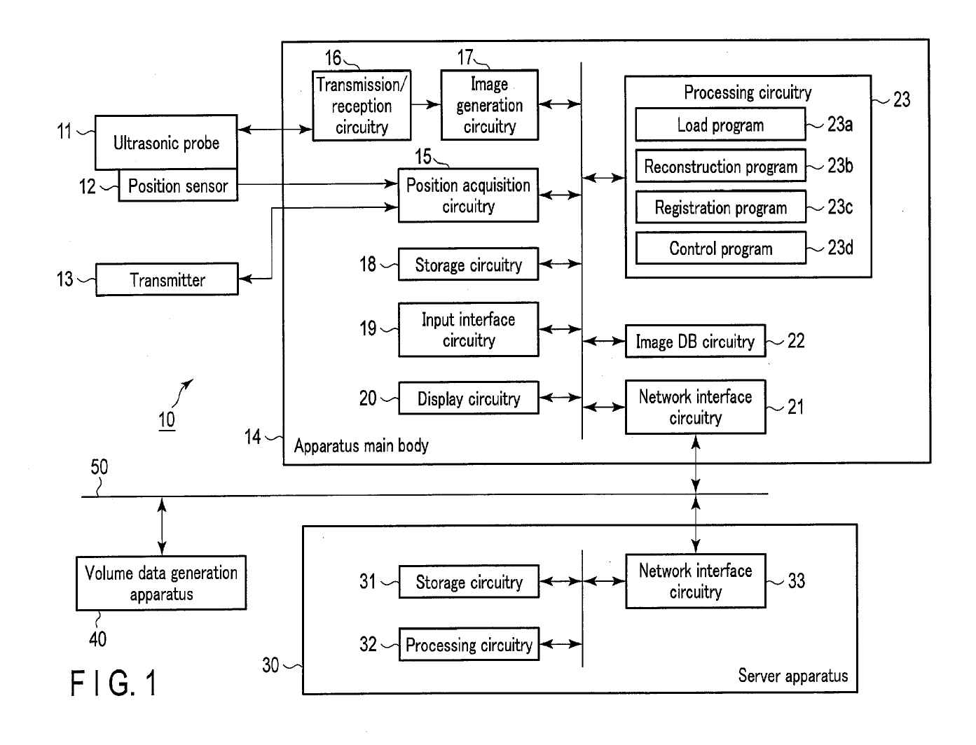

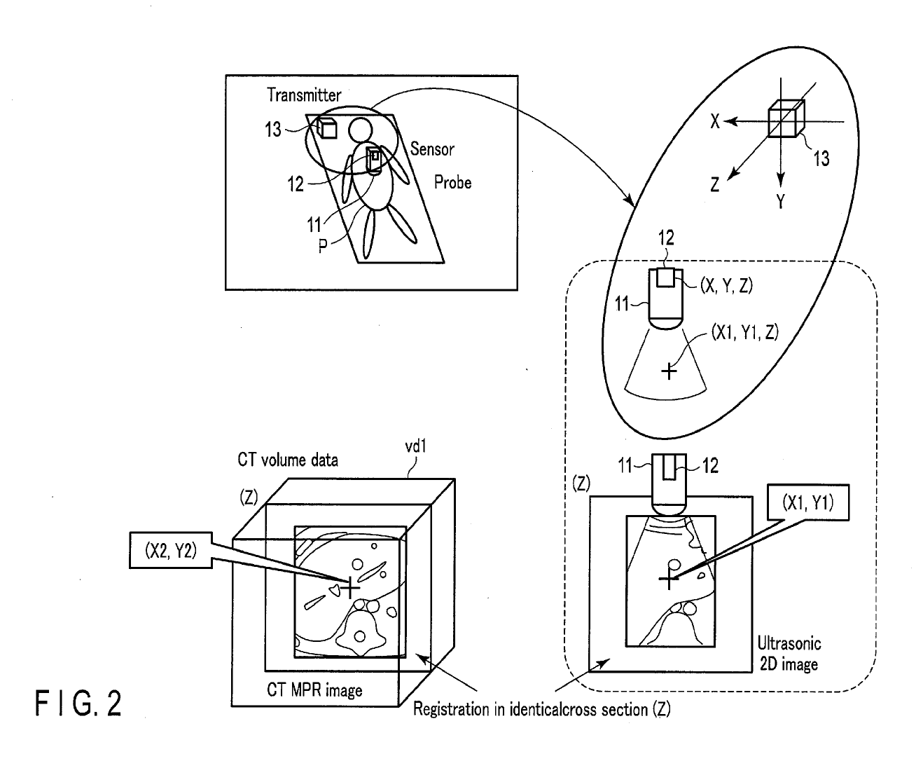

FIG. 1 is a schematic view illustrating an ultrasonic diagnosis apparatus according to a first embodiment, and a peripheral configuration thereof. FIG. 2 is a schematic view for describing a position sensor and a transmitter in this ultrasonic diagnosis apparatus. An ultrasonic diagnosis apparatus 10 is communicably connected to a server apparatus 30 and a volume data generation apparatus 40 over a network 50. Incidentally, the ultrasonic diagnosis apparatus 10 may be connected wirelessly, instead of being connected over the network 50, to the server apparatus 30 and volume data generation apparatus 40.

Here, the ultrasonic diagnosis apparatus 10 includes an ultrasonic probe 11, a position sensor 12, a transmitter 13, and an apparatus main body 14. The apparatus main body 14 includes position acquisition circuitry 15, transmission/reception circuitry 16, image generation circuitry 17, storage circuitry 18, input interface circuitry 19, display circuitry 20, network interface circuitry 21, image DB circuitry 22, and processing circuitry 23.

The ultrasonic probe 11 includes a piezoelectric transducer such as piezoelectric ceramics, which functions as an acoustic/electric reversible conversion element. A plurality of such piezoelectric transducers are juxtaposed, and disposed at a distal end of the ultrasonic probe 11. Incidentally, a description will be given on the assumption that one piezoelectric transducer constitutes one channel. The piezoelectric transducer generates ultrasonic, in response to a driving signal which is supplied from the transmission/reception circuitry 16. The piezoelectric transducer generates a reception echo signal, in response to reception of ultrasonic reflected by a biological tissue of a subject. The ultrasonic probe may be configured as a mechanical four-dimensional probe which executes three-dimensional scan by oscillating a one-dimensional array in a direction perpendicular to the direction of arrangement of plural transducers, or may be configured as a two-dimensional array probe.

The position sensor 12 acquires a position/angle detection signal of the ultrasonic probe 11 with reference to a predetermined reference position. The position/angle detection signal is a detection signal corresponding to the position of the ultrasonic probe 11 and the angle of the ultrasonic probe 11 relative to the predetermined reference position. The angle of the ultrasonic probe 11 is, for example, an inclination of the ultrasonic probe 11 relative to predetermined reference directions. The predetermined reference position is, for example, the position of the ultrasonic diagnosis apparatus 10. The predetermined reference directions are, for example, preset orthogonal three axes. The position sensor 12 is provided, for example, on the ultrasonic probe 11. The position sensor 12 outputs an acquired position/angle detection signal to the position acquisition circuitry 15.

The position sensor 12 is, for example, a magnetic sensor, an infrared sensor, an angle sensor, or an angular velocity sensor (e.g. a gyrosensor). For example, in the case of the magnetic sensor, the magnetic sensor detects magnetism which was transmitted from the transmitter 13, and acquires a position/angle detection signal with reference to the predetermined reference position. In addition, in the case of the infrared sensor, the infrared sensor detects infrared which was transmitted from the transmitter 13, and acquires a position/angle detection signal with reference to the predetermined reference position. Incidentally, general electromagnetic waves may be used in place of the infrared. In the meantime, when the position sensor 12 is the magnetic sensor, the reference position may be the position of the transmitter 13. Additionally, when the position sensor 12 is the infrared sensor, the reference position may be the position of the transmitter 13. Besides, the reference position can be adjusted as needed by an operator's instruction which was input via the input interface circuitry 19. Incidentally, the predetermined reference position may be a position of initial contact with the body surface of the subject.

The angle sensor detects the angle of the ultrasonic probe 11. The angular velocity sensor detects an angular velocity corresponding to the movement of the ultrasonic probe 11. Incidentally, the angle of the ultrasonic probe 11 may be determined based on the positions of two points which are output from, for example, two magnetic sensors, two infrared sensors, or a combination of a magnetic sensor and an infrared sensor, which are provided on side surfaces of the ultrasonic probe 11.

The transmitter 13 is a transmitter which transmits a reference signal that is detected by the position sensor 12. As the transmitter 13, for example, a magnetism transmitter which generates magnetism, or an infrared transmitter which generates infrared can be used as needed.

As illustrated in FIG. 2, the position sensor 12 and transmitter 13 are used for the fusion function of the ultrasonic diagnosis apparatus 10. For example, the position sensor 12 is attached to the ultrasonic probe 11. The transmitter 13 is disposed near a subject P (or in the apparatus main body 14). The position acquisition circuitry 15 (to be described later) calculates the position (X, Y, Z) and angle of the position sensor 12 with reference to the transmitter 13, from the position/angle detection signal which was acquired by the transmission/reception by the transmitter 13 and position sensor 12. Thereby, in the fusion function, the position and angle of the ultrasonic probe 11, which correspond to a currently displayed ultrasonic image, can be obtained. Thereafter, an identical cross section (Z) between the ultrasonic image and an MPR image of other volume data vd1 is displayed, and registration is executed to register position/angle information with respect to an identical position of the identical cross section. In the example illustrated in FIG. 2, a specific position (X1, Y1) in an ultrasonic 2D image and a specific position (X2, Y2) in an MPR image of CT data are set as the identical position.

The position acquisition circuitry 15 calculates the position and angle of the ultrasonic probe 11 with reference to the predetermined position, by using the position/angle detection signal which was output by the position sensor 12. Specifically, the position acquisition circuitry 15 acquires position information including the position and angle of the ultrasonic probe 11 in an absolute coordinate system with reference to the predetermined position. Hereinafter, the position of the ultrasonic probe 11 on the absolute coordinate system is referred to as “probe coordinates”. The position acquisition circuitry 15 sends the acquired position information to the processing circuitry 23. This position acquisition circuitry 15 includes a function of acquiring the position information of the ultrasonic probe 11.

Under the control by the processing circuitry 23, the transmission/reception circuitry 16 supplies driving signals to the respective piezoelectric transducers in the ultrasonic probe 11. The transmission/reception circuitry 16 generates a reception signal, based on reception echo signals which were generated by the respective piezoelectric transducers.

Specifically, the transmission/reception circuitry 16 includes a pulse generator, transmission delay circuitry, pulser circuitry, a preamplifier, an analog-to-digital (hereinafter referred to as “A/D”) converter, reception delay circuitry, and an adder, which are not illustrated.

The pulse generator repeatedly generates rate pulses for forming transmission ultrasonic, at a predetermined rate frequency frHz (cycle: 1/fr second). The generated rate pulses are distributed to a number of channels, and sent to the transmission delay circuitry.

The transmission delay circuitry converges transmission ultrasonic in a beam shape for each of plural channels, and imparts to each rate pulse a transmission delay time which is necessary for determining transmission directivity. The transmission direction of transmission ultrasonic or the transmission delay time (hereinafter referred to as “transmission delay pattern”) is stored in the storage circuitry 18. The transmission delay pattern stored in the storage circuitry 18 is referred to by the processing circuitry 23 when ultrasonic is transmitted.

The pulser circuitry applies a voltage pulse (driving signal) to each of the piezoelectric transducers of the ultrasonic probe, at a timing based on the rate pulse. Thereby, the ultrasonic beam is transmitted to the subject P.

The preamplifier amplifies, with respect to each channel, an echo signal from the subject P which was taken in via the ultrasonic probe 11. The A/D converter converts the amplified reception echo signal to a digital signal.

The reception delay circuitry imparts a reception delay time necessary for determining reception directivity (hereinafter referred to as “reception delay time”) to the reception echo signal which was converted to the digital signal. The reception direction or reception delay time (hereinafter referred to as “reception delay pattern”) of the echo signal is stored in the storage circuitry 18. The reception delay pattern stored in the storage circuitry 18 is referred to by the processing circuitry 23 when ultrasonic is received.

The adder adds the plural echo signals to which the delay time was imparted. By this addition, the transmission/reception circuitry generates a reception signal (also referred to as “RF (radiofrequency) signal”) in which a reflection component from a direction corresponding to the reception directivity is emphasized). By the transmission directivity and reception directivity, the comprehensive directivity of ultrasonic transmission/reception is determined. By this comprehensive directivity, an ultrasonic beam (so-called “ultrasonic scanning line”) is determined.

The image generation circuitry 17 includes a B-mode processing unit and a Doppler processing unit, which are not illustrated. The B-mode processing unit includes an envelope detector and a logarithmic converter, which are not illustrated. The envelope detector executes envelope detection for the reception signal which was output from the transmission/reception circuitry. The envelope detector outputs the envelope-detected signal to the logarithmic converter (to be described later). The logarithmic converter subjects the envelope-detected signal to logarithmic conversion, and relatively emphasizes a weak signal. Based on the signal emphasized by the logarithmic converter, the B-mode processing unit generates a signal value (B-mode data) for each of depths in the transmission/reception of each scanning line and each ultrasonic.

The Doppler processing unit includes a mixer, a low-pass filter, and a velocity/dispersion/power computing device, which are not illustrated. The mixer multiplies the reception signal, which was output from the transmission/reception circuitry 16, by a reference signal having a frequency f0 which is identical to the transmission/reception frequency. By this multiplication, a signal of a component of a Doppler shift frequency fd, and a signal including a frequency component of (2f0+fd) can be obtained. The low-pass filter eliminates the signal of the higher frequency component (2f0+fd) from between the signals including the two kinds of frequency components from the mixer. By eliminating the signal of the higher frequency component (2f0+fd), the Doppler processing unit generates a Doppler signal including the Doppler shift frequency fd.

In the meantime, the Doppler processing unit may use a quadrature detection method in order to generate the Doppler signal. At this time, the reception signal (RF signal) is quadrature-detected, and converted to an IQ signal. By subjecting the IQ signal to complex Fourier transform, the Doppler processing unit generates the Doppler signal including the component of Doppler shift frequency fd. The Doppler signal is, for example, an echo component due to a blood flow, tissue, or contrast medium.

The velocity/dispersion/power computing device includes an MTI (Moving Target Indicator) filter and an autocorrelation computing unit, which are not illustrated. The MTI filter eliminates, from the generated Doppler signal, a Doppler component (clutter component) due to respiratory movement or pulsatory movement of an organ. The autocorrelation computing unit calculates an autocorrelation value for the Doppler signal in which only blood flow information was extracted by the MTI filter. Based on the calculated autocorrelation value, the autocorrelation computing unit calculates a mean velocity value of the blood flow, a dispersion value, and reflection power of the Doppler signal. The velocity/dispersion/power computing device generates color Doppler data, based on a mean velocity value of the blood flow based on plural Doppler signals, a dispersion value, and reflection power of Doppler signals. Hereinafter, the Doppler signal and color Doppler data are comprehensively referred to as “Doppler data”.

In addition, the Doppler data and B-mode data are comprehensively referred to as “raw data”. The raw data may be B-mode data by a high-frequency component of transmission ultrasonic in the echo signal, and elastic data relating to a biological tissue in the subject. The B-mode processing unit and Doppler processing unit output the generated raw data to a digital scan converter (hereinafter referred to as “DSC”) which will be described later. The B-mode processing unit and Doppler processing unit may also output the generated raw data to a cine memory (not shown).

The image generation circuitry 17 includes a DSC (not shown). The image generation circuitry 17 executes a coordinate conversion process (resampling) on the DSC. The coordinate conversion process is a process of converting, for example, a scanning line signal sequence of ultrasonic scan, which is composed of raw data, to a scanning line signal sequence of a general video format, which is typified by television. The image generation circuitry 17 executes on the DSC an interpolation process following the coordinate conversion process. The Interpolation process is a process of interpolating data between scanning line signal sequences by using raw data in neighboring scanning line signal sequences.

By executing the coordinate conversion process and interpolation process on the raw data, the image generation circuitry 17 generates an ultrasonic image as a display image. Incidentally, the image generation circuitry 17 may include an image memory which stores data corresponding to the generated ultrasonic image. The image generation circuitry 17 synthesizes the generated ultrasonic image with character information and scale marks of various parameters. The ultrasonic image generated by using the B-mode data may be called “B-mode image”. In addition, the ultrasonic image generated by using the Doppler data may be called “Doppler image”.

The cine memory is a memory which stores, for example, ultrasonic images corresponding to a plurality of frames immediately before freeze. An ultrasonic motion image can also be displayed by successively displaying (cine display) images stored in this cine memory.

The storage circuitry 18 is composed of memories which store electrical information, such as a ROM (Read Only Memory), a RAM (Random Access Memory), an HDD (Hard Disk Drive) and an image memory, and peripheral circuitry accompanying these memories, such as a memory controller and a memory interface. The storage circuitry 18 stores a plurality of reception delay patterns with different focus depths, a control program of the present ultrasonic diagnosis apparatus, a diagnosis protocol, various kinds of data such as a transmission/reception condition, B-mode data, Doppler data, and B-mode images and Doppler images generated by the image generation circuitry 17.

The input interface circuitry 19 is realized by, for example, a trackball, switch buttons, a mouse, a keyboard, a touchpad which executes an input operation by a touch on an operation screen, and a touch panel display in which a display screen and a touchpad are integrated, these being configured to input to the apparatus main body 14 various instructions, commands, information, selection and settings from the operator. The input interface circuitry 19 is connected to the processing circuitry 23, converts an input operation, which was received from the operator, to an electric signal, and outputs the electric signal to the processing circuitry 23. In the meantime, in this specification, the input interface circuitry 19 is not limited to circuitry including physical operation components such as a mouse and a keyboard. Examples of the input interface circuitry 19 include electric signal processing circuitry which receives an electric signal corresponding to an input operation from an external input device provided separately from the apparatus, and outputs this electric signal to the processing circuitry 23.

The display circuitry 20 is composed of a display which displays medical images, etc., internal circuitry which supplies signals for display to the display, and peripheral circuitry such as connectors and cables which connect the display to the internal circuitry. The display circuitry 20 displays various kinds of ultrasonic images which were generated by the image generation circuitry 17. In addition, the display circuitry 20 may execute, for the ultrasonic image generated by the image generation circuitry 17, adjustment of the brightness, contrast, dynamic range and γ correction, and allocation of a color map. The display circuitry 20 can display an ultrasonic image, and a slice image generated by the processing circuitry 23. Similarly, the display circuitry 20 can interlock-display, by the fusion function of the processing circuitry 23, the ultrasonic image and the MPR image generated by the processing circuitry 23. Specifically, the display circuitry 20 includes a display function of displaying a slice image of volume data, which was reconstructed by the execution of a reconstruction program 23b by the processing circuitry 23 (to be described later), as a reference image, in parallel with the ultrasonic image. In the meantime, as the slice image that is displayed in parallel with the ultrasonic image, for example, an MPR image of CT data, an MPR image of MRI data, or an MPR image of ultrasonic 3D data can be used as needed.

The network interface circuitry 21 is circuitry for connecting the ultrasonic diagnosis apparatus 10 to the network 50, and communicating with the server apparatus 30 and volume data generation apparatus 40. As the network interface circuitry 21, for example, a network interface card (NIC) is usable. In the description below, a description that the network interface circuitry 21 intervenes in the communication between the ultrasonic diagnosis apparatus 10 and the server apparatus 30, etc. is omitted.

The image DB (database) circuitry 22 stores data which was loaded from the server apparatus 30 via the network 50. In addition, the image DB circuitry 22 may include a media drive for reading in data which is stored in an information storage medium such as a USB (universal serial bus) memory, a CD (compact disc) or a DVD (Digital Versatile Disc).

Based on a mode selection, a selection of a reception delay pattern list and the start/end of transmission, which were input by the operator via the input interface circuitry 19, the processing circuitry 23 reads out the transmission/reception condition and apparatus control program stored in the storage circuitry 18, and controls the main body of the ultrasonic diagnosis apparatus. For example, the processing circuitry 23 controls the transmission/reception circuitry 16, position acquisition circuitry 15 and image generation circuitry 17 according to the control program which was read out from the storage circuitry 18. In addition, the processing circuitry 23 executes respective programs 23a to 23d corresponding to functions for realizing the fusion function. Here, the respective programs include, for example, a load program 23a, a reconstruction program 23b, a registration program 23c and a control program 23d.

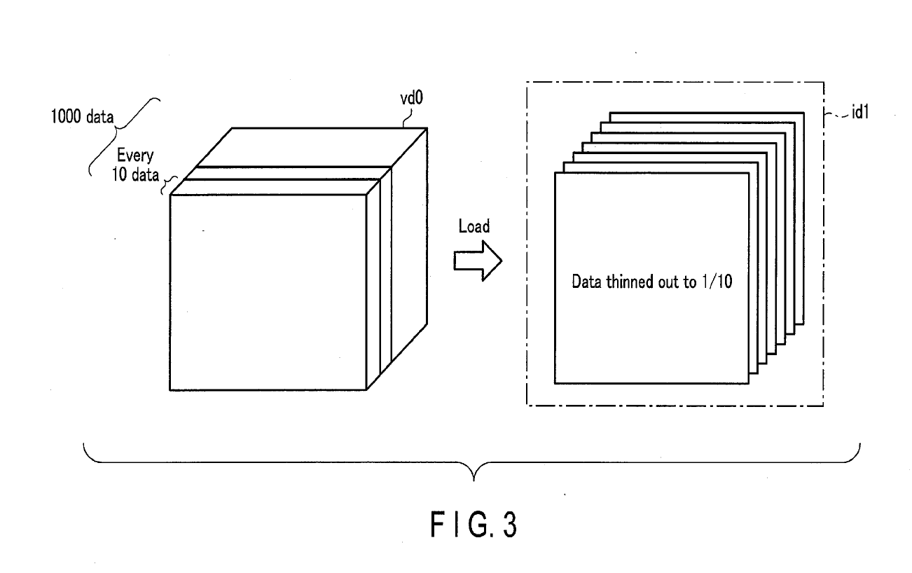

The load program 23a is a program for causing the processing circuitry 23 to execute a load process of loading predetermined data from volume data stored in other apparatus. Here, as the other apparatus, for example, the server apparatus or removable information storage media can be used as needed. In the meantime, as the other apparatus, for example, other modality, such as a CT apparatus, MRI apparatus and nuclear medical diagnosis apparatus, may be used, and a hard disk in the apparatus main body 14 may also be used. In addition, the predetermined data is, for example, partial data of the data which constitute the volume data, and a condition for extracting this partial data is preset. Specifically, for example, the predetermined data may be thinned-out data, data bundled in units of a predetermined number of data, data at a predetermined imaging time, or data of an extracted predetermined region. In the case of the thinned-out data, for example, a condition, such as a read-out interval for thinning-out, is preset. In the case of the data bundled in units of a predetermined number of data, for example, conditions, such as the number of bundled data (number of successively read-out data) and an interval of bundles (read-out interval), are preset. In the case of the data at a predetermined imaging time, for example, a condition, such as an elapsed time (time phase) from the start of imaging, or a date/time of imaging, is preset. The term “time” in the “predetermined imaging time” may be changed, as needed, to some other term, such as “timing” or “time phase”, for specifying time information. In the case of the data of an extracted predetermined region, for example, a condition, such as coordinate values or a size for specifying a predetermined region in volume data, is preset. In addition, as these conditions, for example, predetermined set values may be used, and the predetermined set values may be included in a load request. When the condition is to be changed, the set value indicating the condition may be changed in the load request. Accordingly, for example, the first to fourth load methods can be used, as needed, as the method of loading the predetermined data. As the first load method, for example, as illustrated in FIG. 3, use can be made of a method in which image data, which constitute volume data vd0, are thinned out at regular intervals such that the number of data becomes 1/X, and image data id1 is loaded. However, the thinning-out process is merely an example, and the method is not limited to the thinning-out if the amount of data to be loaded is small and the load time can be reduced. For example, use may be made of a method in which, after the ultrasonic image and the slice image of volume data are registered, image data at a position near the position of the ultrasonic probe 11 is preferentially loaded.

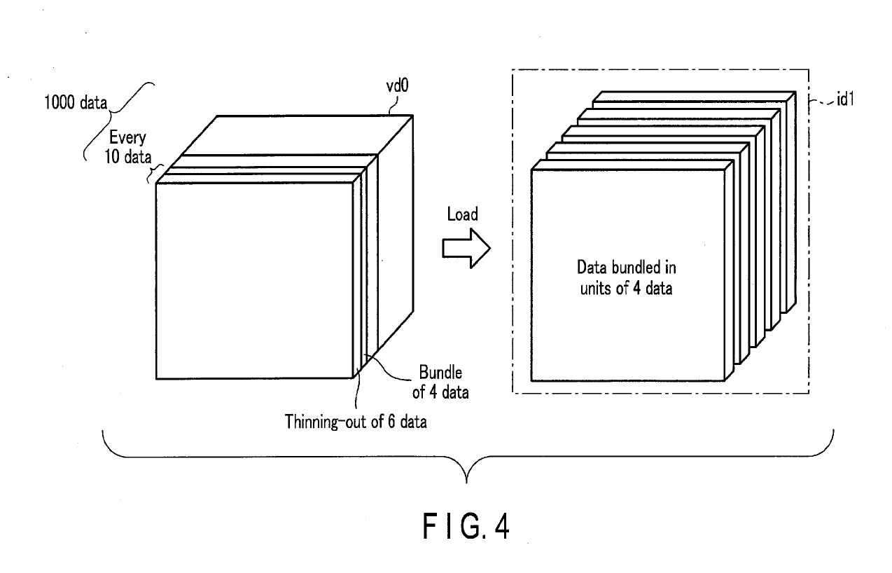

As the second load method, for example, as illustrated in FIG. 4, use can be made of a method of loading data id1 in which image data, which constitute volume data vd0, are bundled in units of a predetermined number of image data. In this case, for example, use can be made of a method of loading data id1 in which image data, which constitute volume data vd0, are bundled in units of a predetermined number of image data at regular intervals, such that the number of the image data, which constitute volume data vd0, becomes 1/X. In the example illustrated in FIG. 4, image data id1, in which every four image data are bundled at intervals of six image data, are loaded.

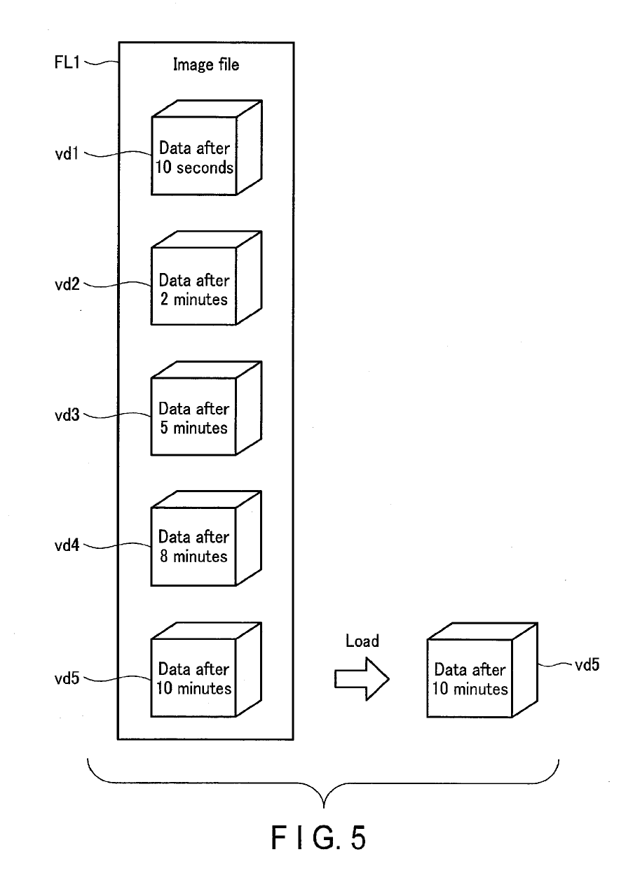

As the third load method, for example, use can be made of a method of loading data of a predetermined imaging time, which is preselected from a plurality of data of different imaging times. For example, in the case of the liver, data of a partial time phase, such as an artery phase or a portal vein phase, can be used as needed. Alternatively, the data of the predetermined imaging time may be follow-up images in the CT apparatus before and after medical treatment. In addition, in the case of the CT apparatus or MRI apparatus, if there are data of three time phases, only the data of one time phase is read in. For example, in the case of the heart, only a telediastoric image, among 4D images, is read in. In addition, in the case of an MRI image or dynamic MRI image, for example, as illustrated in FIG. 5, volume data vd1 to vd5 of five time phases, such as “after 10 seconds”, “after 2 minutes”, “after 5 minutes”, “after 8 minutes” and “after 10 minutes”, are acquired. In DICOM file FL1, five volumes are stored as one file and one series. In the normal fusion function, a DICOM file of one series is read in, and the DICOM file is divided into volume data of five time phases and displayed. By contrast, in the load process of the present embodiment, only the data vd5 of the time phase of “after 10 minutes”, among the data vd1 to vd5 of plural time phases, may be read in. In addition, in the load process of the present embodiment, before loading data, a search may be executed by using a database file such as a DICOMDIR file, and data corresponding to a search result may be loaded. In the case of image data of the heart, end-diastole may be designated by preset, and the DICOMDIR file may be referred to before the DICOM file is read in, and, for example, only image data with a tag of end-diastole at the ascension number may be load.

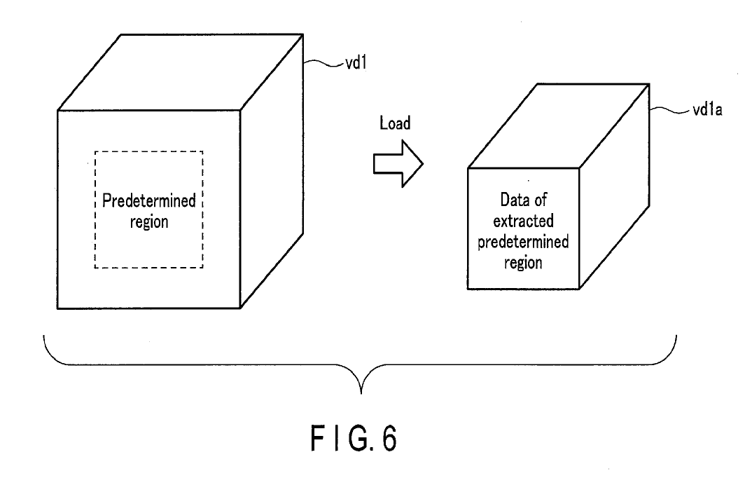

As the fourth load method, use can be made of a method of loading data of an extracted predetermined region from image data which constitute volume data. For example, as illustrated in FIG. 6, data vd1a, which is obtained by extracting a predetermined region with each side of 20 cm that represents the liver, may be loaded from among volume data vd1 of CT images. Alternatively, data obtained by extracting a predetermined liver region from image data, in which the liver and heart are mixedly present, may be loaded. Here, as the predetermined region, use may be made of, as needed, for example, a predetermined central region, a region representing a predetermined organ, a region including the image center and having an area of 30% of the entire data, or a predetermined region of about 60% of the entire data. Incidentally, the ratios of 60% and 30% are merely examples, and the restriction to these ratios is unnecessary. The predetermined region may be referred to as a predetermined partial region. In addition, compared to the process of extracting the region representing the predetermined organ, the process of extracting the predetermined region of about 60% of the entire data does not require a complex process such as recognition of the organ by pattern recognition or the like, and therefore this process can be executed simply and quickly.

Here, a supplemental description is given of the case of thinning out data by selecting a time or a region, as in the third and fourth load methods. In this case, in general, how to thin out data differs depending on the kind of modality. For example, in the case of the data of ultrasonic images, the data amount of even one volume of ultrasonic images is larger than the data amount of one volume of CT images, and therefore the region is thinned out. In the case of CT images or MRI images, either the time phase or the region is thinned out. The reason why how to thin out data differs depending on the kind of modality is that, in general, in the case of CT images or MRI images, the number of time phases increases, compared to the ultrasonic images.

However, as regards the ultrasonic images, if there are data of a plurality of time phases, the time phases can be thinned out. For example, in a contrast agent enhancement method (CHI (Contrast Harmonic Imaging) mode), etc., there is a case in which volume data of ultrasonic images are acquired in accordance with respective time phases, and the volume data of the respective time phases are stored in a DICOM file. In this manner, when the DICOM file of the ultrasonic images includes the volume data of the respective time phases, the time phases can be thinned out, like the third load method.

Besides, load methods other than the first to fourth load methods may be used. For example, in the case of data of PET-CT (Positron Emission computed Tomography-CT), only CT data may be loaded by excluding PET data. In addition, in the case of color Doppler images, either color information or B-mode data may be loaded. Specifically, in the case of double-volume data, only the data of either volume may be loaded.

The reconstruction program 23b is a program which causes the processing circuitry 23 to execute a reconstruction process of reconstructing volume data from the loaded data. In the meantime, a slice image of the reconstructed volume data and an ultrasonic image based on the output of the ultrasonic probe 11 are parallel-displayed on the display circuitry 20.

The registration program 23c is a program which causes the processing circuitry 23 to execute a registration process, so as to register the position of the displayed ultrasonic image and the position of the slice image based on the loaded data.

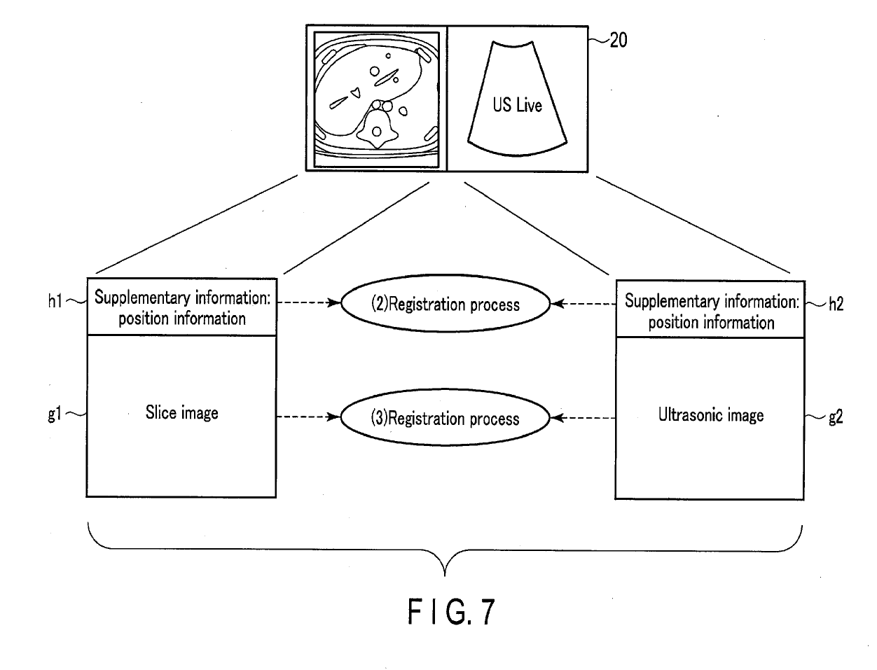

Here, as the registration process, use can be made of, as needed, (1) a registration process by a user's manual operation, (2) a registration process by position information which images have, and (3) a registration process based on images. The registration process (1) is a manual registration process, and includes a process of accepting an operation for registration. Each of the registration processes (2) and (3) is an automatic registration process, and includes a process of executing automatic registration, based on position information which an ultrasonic image g2 and a slice image g1 have, or based on the ultrasonic image g2 and slice image g1, for example, as illustrated in FIG. 7.

For example, the registration process (1) by the user's manual operation successively executes alignment relating to the angle between two images, and registration relating to the position.

The registration process (2) by position information which images have executes registration between two images, based on the position information included in supplementary information (header) h1, h2 of the data displayed on the display circuitry 20. Here, the position information is information indicating a position in the subject, which corresponds to the image of the data. Specifically, the position information includes, for example, coordinate values in the coordinate system of the volume data generation apparatus 40, or probe coordinates acquired by the position acquisition circuitry 15.

The registration process (3) based on images executes registration between two images by comparing the two images. As the registration process based on images, use can be made of, as needed, for example, (3-1) pattern matching, (3-2) point-based registration, and (3-3) surface-based registration.

The pattern matching (3-1) is a method of executing image registration by a similarity calculation between voxels of two images. Thus, the pattern matching may be called “voxel intensity registration”. The scale for measuring the similarity is, for example, a correlation coefficient, or a mutual information amount.

The point-based registration (3-2) is a method of executing image registration by registering a plurality of points existing on two images. As the plural points, use can be made of, as needed, for example, characteristic structures such as branch points of blood vessels.

The surface-based registration (3-3) is a method of executing image registration such that the surfaces of two images become closest in distance. As the surface-based registration, use can be made of, as needed, for example, a Head and Hat method or an ICP (iterative closest point) method.

The control program 23d is a program which causes the processing circuitry 23 to execute, after the registration, a control process of controlling the display circuitry 20 so as to interlock-display the ultrasonic image and slice image in accordance with the position information of the ultrasonic probe 11.

In the meantime, the load process by the load program 23a may include a background load process which loads unloaded residual data on the background, while the ultrasonic probe 11 is being operated. In addition, when the data amount of the loaded partial data is sufficient for an examination, there is no need to load the residual data on the background. Whether the data amount is sufficient or not is determined, for example, based on the threshold of the data amount, etc.

The reconstruction process by the reconstruction program 23b may include an update process of updating the volume data for displaying the slice image, by the data loaded on the background and the loaded partial data.

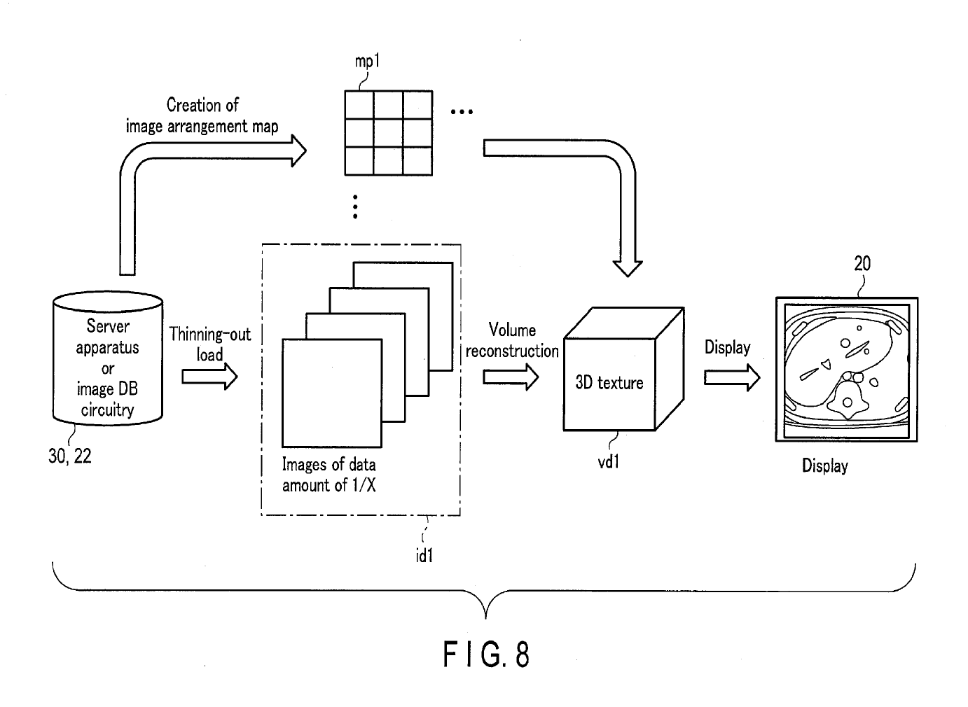

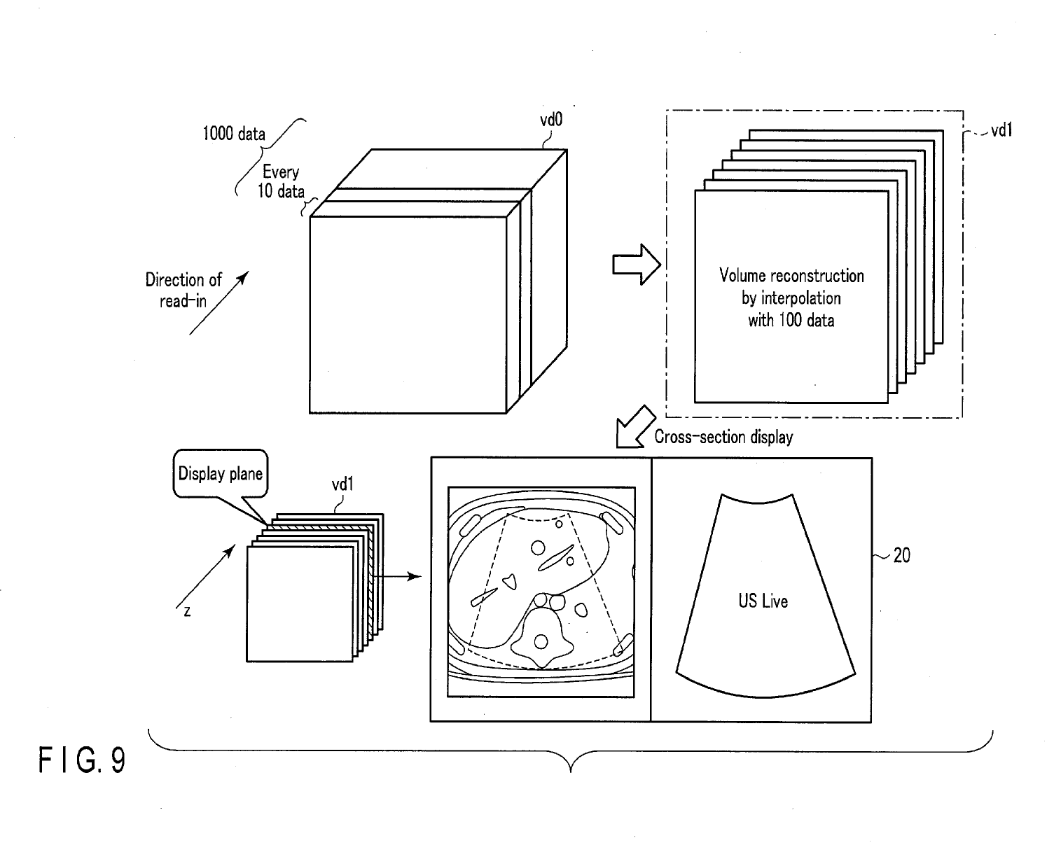

Additionally, the volume data may be constructed of a plurality of image data (2D data). The load process by the load program 23a may load, as predetermined data, partial image data which is obtained by thinning out the plural image data. In the case of an example illustrated in FIG. 8, by executing the load program 23a, the processing circuitry 23 loads image data id1 of a data amount of 1/x, which is obtained by thinning out the image data in the volume data in the server apparatus 30 or image DB circuitry 22, and creates an image arrangement map mp1. The image arrangement map mp1 is a map for creating a 3D texture, and is used even when thinning-out is not executed. As the image arrangement map mp1, use can be made of, as needed, for example, a list in which a slice number (identification information of image data) can be written for each read-out position, or a multidimensional map in which data identification information can be written in accordance with read-out positions. However, the image arrangement map mp1 is not limited to the above-described list or multidimensional map, and an arbitrary structure may be used as needed if the structure in which identification information and read-out positions of image data can be recorded. In addition, by executing the reconstruction program 23b, the processing circuitry 23 reconstructs the volume data vd1 of the 3D texture from the loaded image data id1, based on the created image arrangement map mp1, and displays the slice image of the volume data vd1 on the display circuitry 20. In the case of an example illustrated in FIG. 9, one image data in every 10 image data is read in from the image DB circuitry 22 which stores volume data vd0 that is composed of 1000 image data. Specifically, 100 image data of 1000 image data, which constitute the volume data vd0, are read in the processing circuitry 23. The processing circuitry 23 executes an interpolation process on the read-in 100 image data, thereby reconstructing the volume data vd1. Then, the processing circuitry 23 displays, on the display circuitry 20, the slice image of the volume data vd1 and the ultrasonic image (indicated as “US Live” in FIG. 9).

In the embodiment of FIG. 1, the respective functions, which are executed in the processing circuitry 23, are stored in the storage circuitry 18 in the form of computer-executable programs. The processing circuitry 23 is a processor which reads out the programs from the storage circuitry 18 and executes the programs, thereby realizing the functions corresponding to the respective programs. In other words, the processing circuitry in the state in which the processing circuitry has read out the programs includes the programs 23a to 23d indicated in the processing circuitry 23 in FIG. 1. In the meantime, in FIG. 1, the description was given on the assumption that the respective functions are realized by the single processing circuitry 23. However, the processing circuitry may be constructed by combining a plurality of independent processors, and the respective functions may be realized by the respective processors executing the programs.

The server apparatus 30 includes storage circuitry 31, processing circuitry 32 and network interface circuitry 33.

The storage circuitry 31 stores volume data which was transferred from other modality such as the volume data generation apparatus 40. In addition, the storage circuitry 31 may store volume data which was transferred from, aside from the other modality, the same modality (ultrasonic diagnosis apparatus 10) as the modality which generates the ultrasonic image.

The processing circuitry 32 includes a function of instructing the volume data generation apparatus 40 to execute data transfer, and writing volume data, which was transferred from the volume data generation apparatus 40, into the storage circuitry 31. In addition, the processing circuitry 32 includes a read-out function of reading out predetermined data from the volume data of the storage circuitry 31, based on a load request transmitted from the ultrasonic diagnosis apparatus 10. Here, the predetermined data is, for example, partial data of the data which constitute the volume data. Furthermore, the processing circuitry 32 includes a first return function of returning the read-out data to the source of transmission of the load request.

In the embodiment of FIG. 1, the respective functions, which are executed in the processing circuitry 32, are stored in the storage circuitry 31 in the form of computer-executable programs. The processing circuitry 32 is a processor which reads out the programs from the storage circuitry 31 and executes the programs, thereby realizing the functions corresponding to the respective programs. In other words, the processing circuitry 32 in the state in which the processing circuitry 32 has read out the programs includes the programs corresponding to the respective functions. In the meantime, in FIG. 1, the description was given on the assumption that the respective functions are realized by the single processing circuitry 32. However, the processing circuitry may be constructed by combining a plurality of independent processors, and the respective functions may be realized by the respective processors executing the programs.

The network interface circuitry 33 is circuitry for connecting the server apparatus 30 to the network 50, and communicating with the ultrasonic diagnosis apparatus 10 and volume data generation apparatus 40. As the network interface circuitry 33, for example, a network interface card (NIC) is usable. In the description below, a description that the network interface circuitry 33 intervenes in the communication between the server apparatus 30, on one hand, and the ultrasonic diagnosis apparatus 10 and volume data generation apparatus 40, on the other hand, is omitted.

The volume data generation apparatus 40 is an apparatus which generates volume data by scanning the subject. As the volume data generation apparatus 40, use can be made of, as needed, for example, arbitrary modality such as an ultrasonic diagnosis apparatus, CT apparatus, MRI apparatus and nuclear medical diagnosis apparatus. The volume data generation apparatus 40 transfers volume data to the server apparatus 30 via the network 50 in accordance with an instruction from the server apparatus 30.

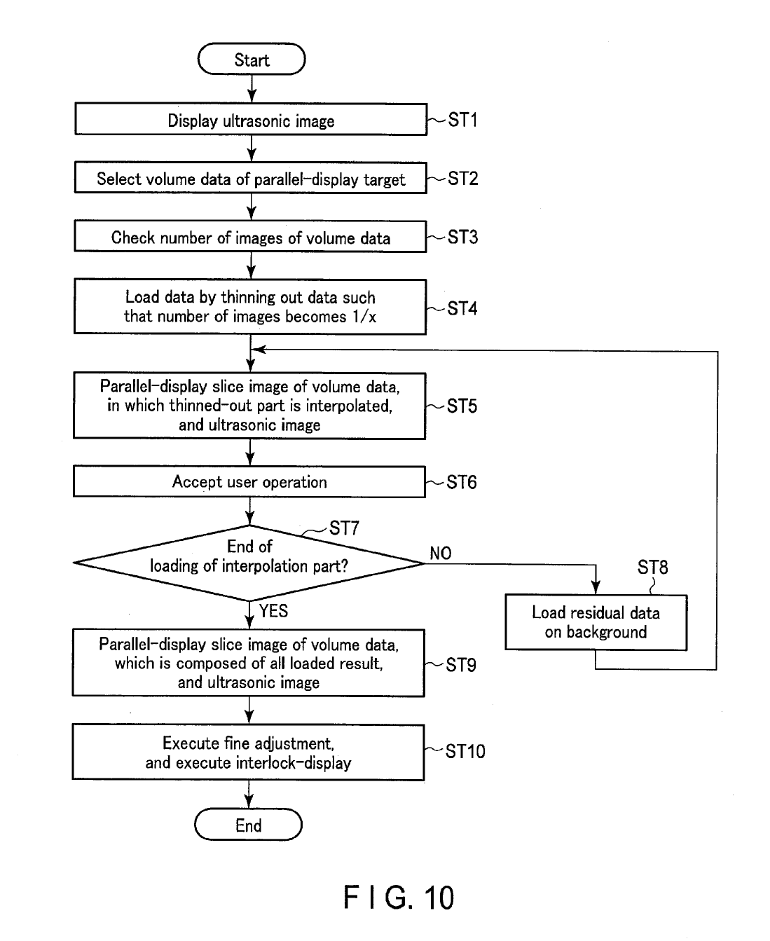

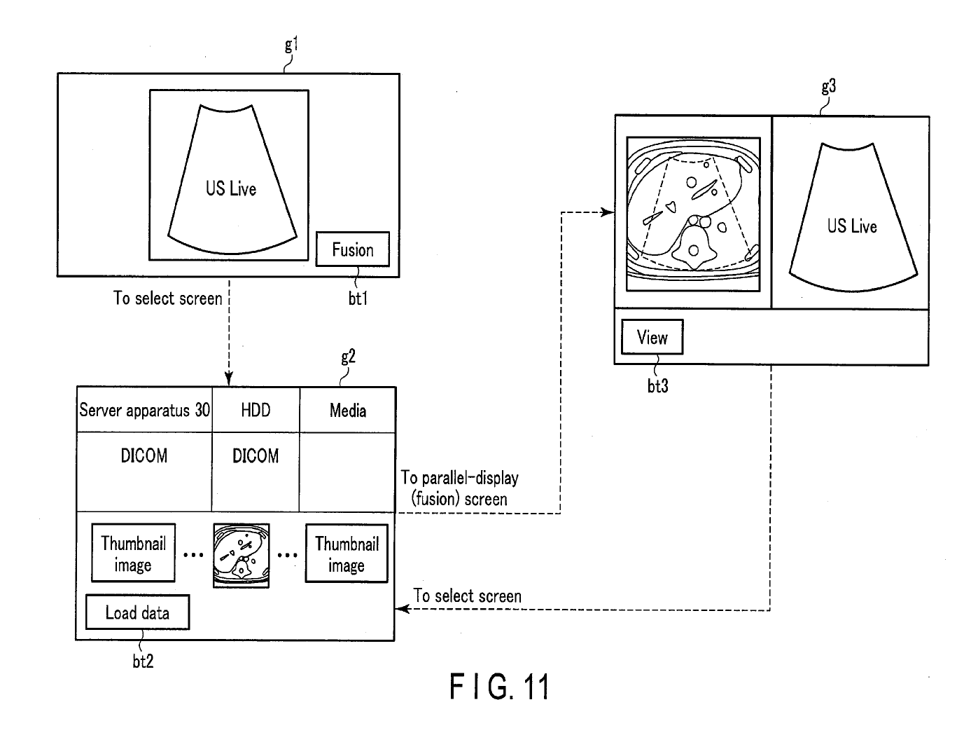

Next, the operation of the ultrasonic diagnosis apparatus having the above-described configuration will be described with reference to a flowchart of FIG. 10 and a schematic view of FIG. 11. Incidentally, the description below is given by taking as an example the case in which thinned-out data of loadable data is loaded as predetermined data. However, the predetermined data is not limited to the thinned-out data. For example, data bundled in units of a predetermined number of data, data at a predetermined imaging time, or data of an extracted predetermined region may be loaded. Alternatively, only the data of one volume of double-volume data may be loaded. In addition, in the description below, the case of executing the registration process (1) of the above-described registration processes (1) to (3) is taken as an example. However, the registration process is not limited to (1), and the above-described registration process (2) or (3) may be executed. Thus, the “predetermined data” and “registration process” in the description below are merely examples, and other data and other registration processes may be implemented, as needed, also in the embodiments to be described below. Next, the operation of the ultrasonic diagnosis apparatus 10 will be described.

In the ultrasonic diagnosis apparatus 10, by an operation of the user such as a doctor, the ultrasonic probe 11 is put in contact with a subject, the inside of the subject is scanned by an ultrasonic beam, and an ultrasonic image is generated based on an output of the ultrasonic probe 11. Thereby, in the ultrasonic diagnosis apparatus 10, as illustrated in FIG. 11, a display screen g1 including an ultrasonic image and a “Fusion” button bt1 is displayed on the display circuitry 20 (step ST1). By a user's operation of pressing the “Fusion” button bt1, the processing circuitry 23 of the ultrasonic diagnosis apparatus 10 displays, on the display circuitry 20, a select screen g2 for selecting volume data that is a parallel-display target, in place of the display screen g1. It is assumed that load sources, such as the server apparatus 30, HDD and information storage media, are first displayed on the select screen g1.

For example, if the server apparatus 30 is designated as a load source by the user's operation on the select screen g2, the processing circuitry 32 displays, on the select screen g2, thumbnail images of slice images of volume data stored in the designated load source.

It is assumed that one of the thumbnail images was designated from among the displayed thumbnail images by the user's operation, and a “Load data” button bt2 was pressed. Thereby, the processing circuitry 23 selects volume data corresponding to the designated thumbnail image (step ST2), and checks the number of images of the volume data (step ST3).

Subsequently, the processing circuitry 23 transmits to the server apparatus 30 a load request including identification information of the volume data and the read-out position and read-out intervals of image data in the volume data, so as to load the data by thinning out the data such that the number of images becomes 1/x.

Based on this load request, the server apparatus 30 transmits partial data of the volume data to the ultrasonic diagnosis apparatus 10. Specifically, based on the read-out position and read-out intervals, the server apparatus 30 reads out reference image data, which serves as a reference, from the read-out position in the volume data, and writes the identification information of the reference image data in the image arrangement map, based on the read-out position. In addition, the server apparatus 30 reads out image data at read-out intervals from the reference image data, and writes the identification information of the image data in the image arrangement map, based on the read-out position of the image data. Thereafter, the server apparatus 30 successively transmits the image arrangement map and each image data to the ultrasonic diagnosis apparatus 10.

The processing circuitry 23 of the ultrasonic diagnosis apparatus 10 writes the received image arrangement map and each received image data in the storage circuitry 18 (step ST4). These steps ST2 to ST4 are an example of the load process which is realized by the processing circuitry 23 reading out the load program 23a from the storage circuitry 18 and executing the load program 23a. The load process is a process of loading predetermined data from the volume data stored in other apparatus.

After the completion of reception, the processing circuitry 23 reconstructs volume data in which the thinned-out part (the part of 1−1/x) is interpolated, based on the image arrangement map and each image data in the storage circuitry 18. Thereafter, the processing circuitry 23 parallel-displays the slice image of the volume data and the ultrasonic image on the display circuitry 20 (step ST5). In the example of a fusion screen g3 illustrated in FIG. 11, the slice image of the volume data and the ultrasonic image are arranged in parallel in the left-and-right direction. In the meantime, if the thumbnail image was erroneously designated and the volume data is to be re-selected, a “View” button bt3 in the fusion screen g3 is press-operated. Thus, a transition occurs to the select screen g2, and the process of step ST2 onwards is executed once again. The reconstruction in step ST5 is an example of the reconstruction process which is realized by the processing circuitry 23 reading out the reconstruction program 23b from the storage circuitry 18 and executing the reconstruction program 23b. The reconstruction process is a process of reconstructing volume data from the data loaded by the load process.

After the start of parallel-display by step ST5, the processing circuitry 23 transitions to a user operation acceptance state for accepting a user operation (step ST6). This step ST6 is an example of the registration process which is realized by the processing circuitry 23 reading out the registration program 23c from the storage circuitry 18 and executing the registration program 23c. The registration process is a process which is executed so as to register the positions of the ultrasonic image and slice image which are displayed by the display circuitry 20. In the user operation acceptance state, to begin with, registration of position/angle information between the slice image of volume data and the ultrasonic image is executed by the user's operation. This registration is executed in the order of alignment relating to the angle, and registration relating to the position.

Specifically, in the ultrasonic diagnosis apparatus 10, the ultrasonic probe 11, which is put in contact with the subject, is operated, and the position information of the ultrasonic probe 11 is acquired by the position acquisition circuitry 15. Using the angle which is indicated by the position information, the processing circuitry 23 executes registration (alignment) between the orthogonal three axes of the position coordinate system relating to the ultrasonic probe 11 and the orthogonal three axes of the coordinate system in the volume data.

Subsequently, the ultrasonic diagnosis apparatus 10 acquires the position information of the ultrasonic probe 11 in accordance with the operation of the ultrasonic probe 11. Using the position indicated by the acquired position information, the processing circuitry 23 executes registration between the reference point of the position coordinate system relating to the ultrasonic probe 11 and the reference point of the coordinate system in the volume data. Thereby, the registration is completed. In the meantime, in the first registration, complete registration is not always necessary, since there is a case in which fine adjustment is executed in step ST10 (to be described later).

Next, in the user operation acceptance state, the ultrasonic image and the slice image of the volume data, which have been registered, are interlock-displayed on the display circuitry 20. This interlock-display is an example of the control process which is realized by the processing circuitry 23 reading out the control program 23d from the storage circuitry 18 and executing the control program 23d. This control process is a process of controlling the display circuitry 20 so as to interlock-display the ultrasonic image and slice image in accordance with the position information of the ultrasonic probe 11.

Specifically, the ultrasonic diagnosis apparatus 10 generates the ultrasonic image in accordance with the operation of the ultrasonic probe 11. The ultrasonic diagnosis apparatus 10 generates the slice image (MPR image) corresponding to the ultrasonic image, based on the registered volume data. Thereby, the ultrasonic diagnosis apparatus 10 interlock-displays the ultrasonic image and slice image of the volume data in accordance with the position of the ultrasonic probe 11.

Thereafter, in the user operation acceptance state, while the user visually recognizes the ultrasonic image and slice image of the volume data which are interlock-displayed, the user applies puncture or RFA treatment to the subject.

On the other hand, in the user operation acceptance state of step ST6, while the processing circuitry 23 accepts a user operation, the processing circuitry 23 determines whether the loading of the interpolation part is to be ended or not (step ST7). For example, if a load end instruction is not input and there is unloaded residual data, the processing circuitry 23 determines “NO”, transmits a load request in the background, loads the residual data (step ST8), and returns to step ST5. In step ST5 of the second and following times, the processing circuitry 23 reconstructs and updates the volume data for displaying the slice image, by using the residual data loaded on the background and the loaded partial data. Thereafter, the processing circuitry 23 parallel-displays the slice image of the updated volume data and the ultrasonic image on the display circuitry 20.

In the meantime, the load end instruction is input to the ultrasonic diagnosis apparatus 10 by the user operation, for example, when the slice image of the volume data reconstructed from the loaded data is sufficient for puncture or RFA treatment. Specifically, the load end instruction is used for stopping further loading, when puncture or RFA treatment can sufficiently be performed with the loaded partial data of the volume data.

In addition, in the determination of step ST7, for example, if the load end instruction is input by the user operation or if the loading of all data is completed, the ultrasonic diagnosis apparatus 10 determines that the loading is to be ended, and transitions to step ST9.

In step ST9, the processing circuitry 23 parallel-displays on the display circuitry 20 the slice image of the volume data, which was reconstructed from all loaded results, and the ultrasonic image.

During this parallel-display, the processing circuitry 23 finely adjusts the alignment result and registration result between the slice image of the volume data and the ultrasonic image in accordance with the user operation, and executes interlock-display (step ST10). Thereafter, in the same manner as described above, while the user visually recognizes the ultrasonic image and slice image of the volume data which are interlock-displayed, the user applies puncture or RFA treatment to the subject.

As has been described above, according to the present embodiment, predetermined data is loaded from the volume data stored in other apparatus, and the volume data is reconstructed from the loaded data. In addition, the slice image of the reconstructed volume data and the ultrasonic image, which is based on the output of the ultrasonic probe, are displayed in parallel.

Thus, according to this embodiment, the load time of volume data can be shortened, compared to the case of loading all data in the volume data. Therefore, a hindrance to the workflow due to the load time of volume data can be reduced. For example, since the load time can be shortened, a step of a target search or registration after data loading can be started earlier, and therefore the throughput time of puncture or RFA treatment can be shortened.

Additionally, since the load time is shortened, the timing of determining whether the loaded data is desired volume data or not comes earlier. Thus, even when new data is reloaded in accordance with the determination result that the loaded data is not desired volume data, a loss time due to loading of undesired data can be decreased.

A supplemental description is given. In conventional data load quickening techniques, since the load time cannot be shortened when data is first loaded, the problem remains unsolved. In addition, in the thumbnail display or comment function, the necessary information for puncture or RFA treatment is deficient, and there is a case in which part of the necessary information is first understood by parallel-displaying the MPR image. On the other hand, there is a case in which, after all data in volume data is loaded and an MPR image thereof is parallel-displayed, it is determined that the loaded data is not the desired data. In this case, a need arises to reload all data in other volume data, and a load time is consumed once again.

However, according to the present embodiment, since the load time is shortened, the timing of determining whether the loaded data is desired volume data or not comes earlier, and therefore a loss time due to loading of undesired data can be decreased.

Additionally, according to this embodiment, while the ultrasonic probe 11 is being operated, the unloaded residual data is loaded on the background. In addition, the volume data for displaying the slice image is updated by the residual data, which was loaded on the background, and the loaded partial data. Accordingly, since the residual data is loaded on the background during the operation of the ultrasonic probe 11 for registration and interlock-display, the throughput time of puncture or RFA treatment can be shortened.

Additionally, the image quality is important for the slice image which is interlock-displayed after the registration. Thus, the image quality is made higher than at the time of the registration, by loading the residual data and updating the volume data. On the other hand, there is no problem even if the image quality is low for the slice image which is displayed at the time of registration, and thus the load time is shortened by loading partial data. Specifically, slice images with different image qualities can be used in accordance with the purpose of use, for example, by displaying a slice image with low image quality for registration, and displaying a slice image with high image quality for puncture or RFA treatment. Furthermore, a slice image, which has a higher image quality in step with the enhancement in resolution of medical diagnosis apparatuses in recent years, can be guide-displayed for puncture or RFA treatment.

Additionally, although the operation of the first embodiment was described by taking the registration by the manual operation as an example, the restriction to this is unnecessary and automatic registration may be executed. In the automatic registration method, registration is executed between the slice image of the volume data, which was acquired by loading and reconstruction, and the ultrasonic image. At the time of the automatic registration, a GPU (Graphics Processing Unit) may preferably be used as a part of the processing circuitry 23 in order to increase the speed of calculations. However, if full-volume data is read in the memory of the GPU, there may be a case in which the data cannot completely be read in the memory because the memory capacity of the GPU is limited and the memory capacity is deficient. Thus, by the above-described load process, the data amount of the volume data can be reduced and an allowance can be given to the memory capacity of the GPU. Thereby, in addition to the increase in speed of loading, the resources, such as the memory capacity of the GPU, can be saved. Similarly, when the fusion function is also used for volume data including Doppler-based color information, the data amount increases by an amount of color information, there arises a problem that the resource deficiency tends to easily occur. To cope with this, the resource deficiency can be prevented, for example, by selectively thinning out information corresponding to a specific color from the color information corresponding to a plurality of colors, and thus reducing the data amount of the color information.

### Second Embodiment

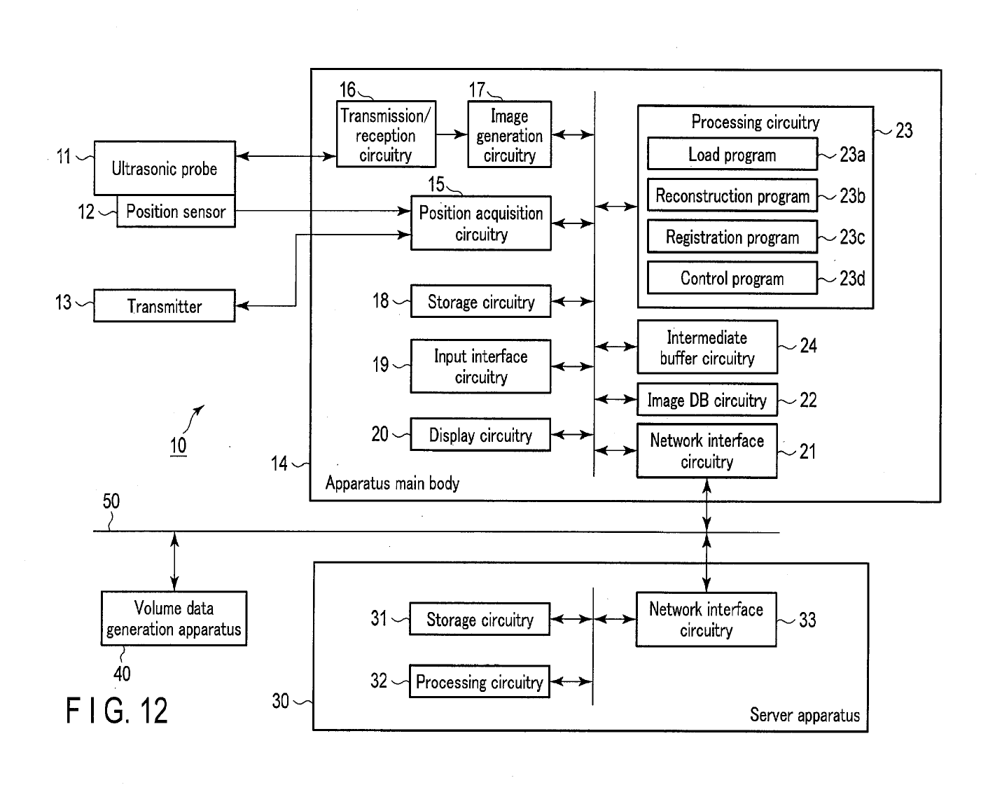

FIG. 12 is a schematic view illustrating an ultrasonic diagnosis apparatus according to a second embodiment, and a peripheral configuration thereof. The parts, which are substantially identical to those in FIG. 1, are denoted by like reference numerals, and a detailed description thereof is omitted, and only different parts will mainly be described here. As regards the respective embodiments to be described below, an overlapping description will similarly be omitted.

The second embodiment is a concrete example of the first embodiment. As illustrated in FIG. 12, compared to the configuration shown in FIG. 1, the second embodiment includes intermediate buffer circuitry 25.

The intermediate buffer circuitry 25 is storage circuitry which temporarily stores data that is loaded on the background.

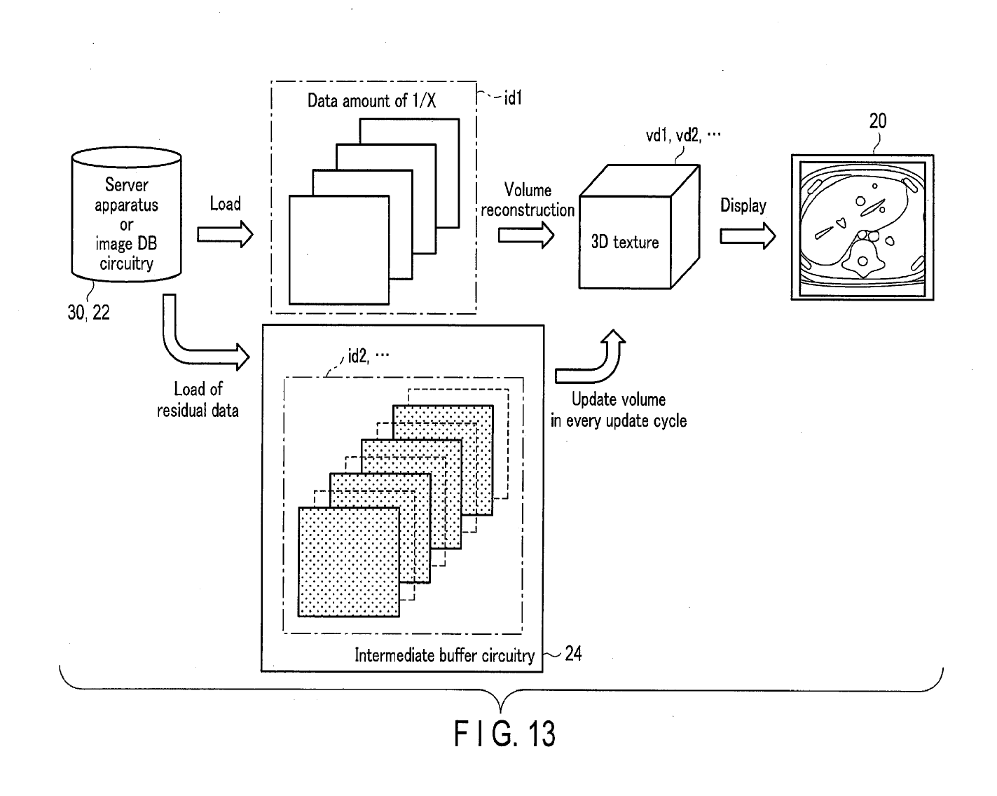

Accordingly, the load process by the load program 23a of the processing circuitry 23 includes a background load process of loading unloaded residual data on the background, while the ultrasonic probe 11 is being operated. For example, as illustrated in FIG. 13, the processing circuitry 23 loads, in like manner as described above, image data id1 of a data amount of 1/x, from among the volume data in the server apparatus 30 or image DB circuitry 22. In addition, in the same manner as described above, the processing circuitry 23 reconstructs the volume data of the 3D texture from the loaded image data id1, and displays the slice image of the volume data vd1 on the display circuitry 20. Thereafter, by the background process, the processing circuitry 23 loads residual image data id2, . . . , on the background, and stores the image data id2, . . . , in the intermediate buffer circuitry 24.

The reconstruction process by the reconstruction program 23b of the processing circuitry 23 includes an update process of updating the volume data for displaying the slice image, by the residual data loaded on the background and the loaded partial data. For example, in the case of the example of FIG. 13, by the update process, the processing circuitry 23 reconstructs volume data vd2 of the 3D texture by the image data id2 loaded in the intermediate buffer circuitry 24 on the background, and the already loaded image data id1. Then, the processing circuitry 23 updates the volume data vd1, which was reconstructed previously, to the volume data vd2 which was reconstructed this time. Thereby, compared to the volume data vd1 which was reconstructed previously, the volume data vd2 reconstructed this time has an enhanced image quality of a display plane perpendicular to the z axis by a degree corresponding to the image data id2 loaded this time. Update by this update process is repeatedly executed, for example, in predetermined update cycles. For example, in the case of the update of the next time, the processing circuitry 23 reconstructs volume data vd3 of the 3D texture by image data id3 loaded in the intermediate buffer circuitry 24 on the background, and the already loaded image data id1 and id2. Then, the processing circuitry 23 may update the volume data vd2, which was reconstructed previously, to the volume data vd3 which was reconstructed this time.

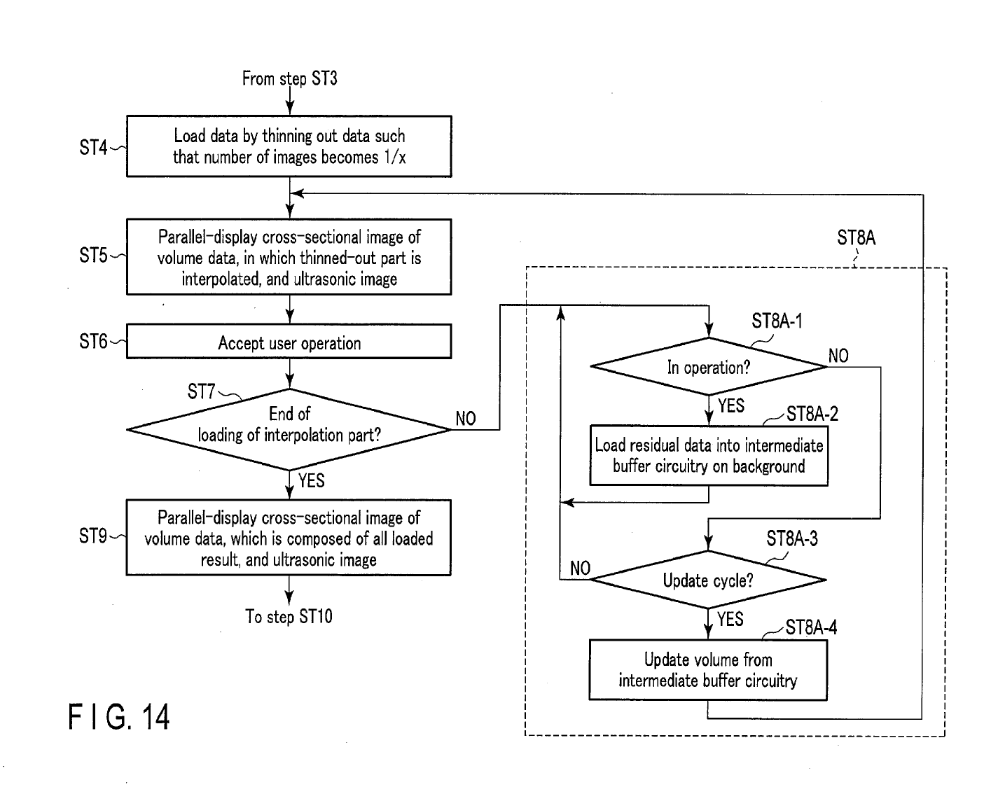

Next, the operation of the ultrasonic diagnosis apparatus with the above-described configuration will be described with reference to a flowchart of FIG. 14.

Now, steps ST1 to ST7 are executed in the same manner as described above. In step ST7, if it is determined that the loading of the interpolation part is not to be ended, the ultrasonic diagnosis apparatus 10 executes, in place of the above-described step ST8, step ST8A which includes ST8A-1 to ST8A-4.

Specifically, the processing circuitry 23 determines whether the user is operating the input interface circuitry 19 (step ST8A-1). If the determination result of step ST8A-1 indicates that the user is operating the input interface circuitry 19, the processing circuitry 23 transmits a load request to the server apparatus 30 on the background, loads residual data from the server apparatus 30 into the intermediate buffer circuitry 24 (step ST8A-2), and returns to step ST8A-1.

On the other hand, if the determination result of step ST8A-1 indicates that the user is not operating the input interface circuitry 19, the processing circuitry 23 determines whether a predetermined update cycle has come or not (step ST8A-3). If “NO” in step ST8A-3, the processing circuitry 23 returns to step ST8A-1.

If the determination result of step ST8A-3 indicates that the predetermined update cycle has come, the processing circuitry 23 reconstructs volume data by the image data loaded in the intermediate buffer circuitry 24 on the background, and the already loaded image data. Then, the processing circuitry 23 updates the volume data, which was reconstructed previously, to the volume data which was reconstructed this time (step ST8A-4), and advances to step ST5.

On the other hand, in step ST7, if it is determined that the loading of the interpolated part is to be ended, the ultrasonic diagnosis apparatus 10 executes the process of step ST9 onwards in the manner as described above.

As described above, according to the present embodiment, while the ultrasonic probe 11 is being operated, the unloaded residual data is loaded in the intermediate buffer circuitry 24 on the background. In addition, the volume data for displaying the slice image is updated by the residual data loaded on the background and the loaded partial data.

Thus, according to the present embodiment, by the configuration including the intermediate buffer circuitry 24 which temporarily stores residual data that is loaded on the background, the load process of data and the update process of volume data do not interfere with each other. Thereby, in addition to the advantageous effects of the first embodiment, it becomes easier to execute the storage of data on the background, the reconstruction and the update.

A supplemental description is given. The processing circuitry 23 progresses the loading of data in the intermediate buffer circuitry 24 while the ultrasonic probe 11 is being operated. In addition, when the ultrasonic probe 11 is not being operated, the processing circuitry 23 reconstructs volume data from the data in the intermediate buffer circuitry 24, and updates the volume data. Accordingly, the loading of data and the update of volume data can be switched and executed, depending on whether the ultrasonic probe 11 is being operated or not.

Additionally, according to the present embodiment, since the reconstructed volume data is updated in update cycles, the image quality of volume data can be improved stepwise in accordance with the update cycles.

### Third Embodiment

Next, an ultrasonic diagnosis apparatus according to a third embodiment will be described with reference to FIG. 1.

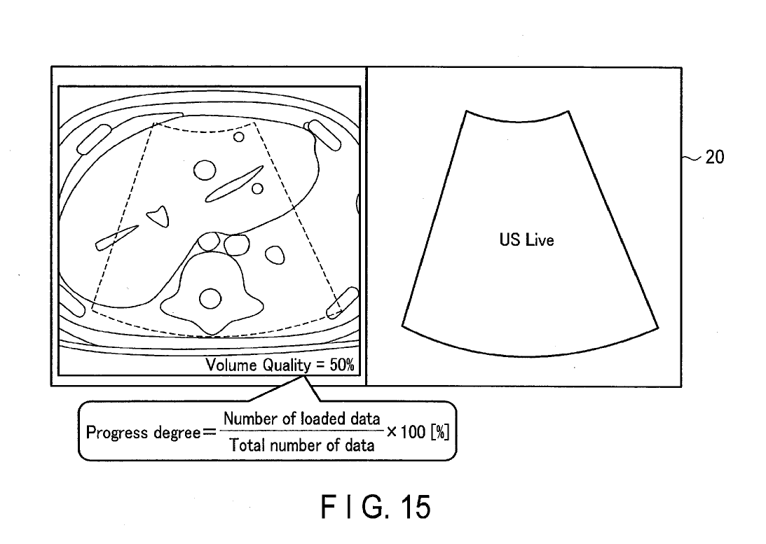

The third embodiment is a modification of the first embodiment, and has such a configuration that the progress degree of loading is displayed on the display circuitry 20, as illustrated in FIG. 15. Although an equation in FIG. 15 is not displayed on the actual screen, the equation may be displayed when the cursor is moved to the vicinity of a progress degree “Volume Quality=50%”. In addition, since the progress degree corresponds to the image quality of volume data, the progress degree may be displayed as “Volume Quality”. Specifically, the term “progress degree” may be replaced with another term if the term expresses the corresponding meaning.

Accordingly, the control process by the control program 23d of the processing circuitry 23 calculates the progress degree of loading by the background load process, and, after the update by the update process, controls the display circuitry 20 so as to further display the calculated progress degree. The progress degree can be calculated, for example, as the ratio of the number of loaded images to the total number of images.

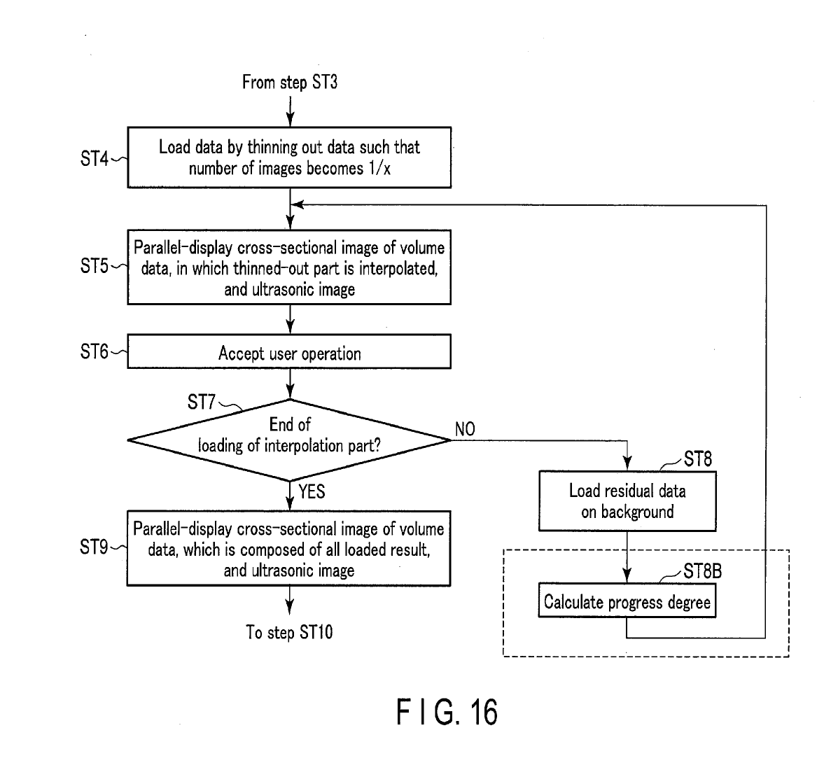

Next, the operation of the ultrasonic diagnosis apparatus with the above-described configuration will be described with reference to a flowchart of FIG. 16.

Now, steps ST1 to ST8 are executed in the same manner as described above. After the completion of step ST8, the processing circuitry 23 calculates the progress degree of loading by the background load process (step ST8B), and goes to step ST5. In step ST5 of the second and following times, the processing circuitry 23 updates the volume data by the update process, and then controls the display circuitry 20 so as to parallel-display the slice image of volume data and the ultrasonic image, and to further display the progress degree calculated in step ST8B. Specifically, since the progress degree is displayed after the update of volume data, the progress degree serves as an index corresponding to the image quality of the slice image of the updated volume data.

Subsequently, the steps of step ST6 onwards are executed in the same manner as described above.

As described above, according to the present embodiment, the progress degree of loading is calculated, and, after the volume data is updated, the calculated progress degree is displayed. By this configuration, in addition to the advantageous effects of the first embodiment, the index corresponding to the image quality of the slice image (MPR image), which is being displayed, can be presented to the user.

A supplemental description is given. When the data load is progressing on the background, the volume data reconstructed from the partial data is incomplete volume data. Thus, by displaying a GUI (Graphical User Interface) for presenting the progress degree of loading to the user, it becomes possible to present to the user the progress degree of data loading on the background and the image quality of the slice image that is being displayed.

In the meantime, although the third embodiment was described as being implemented as the modification of the first embodiment, the restriction to this is unnecessary, and the third embodiment may be implemented as a modification of the second embodiment. Thereby, in addition to the advantageous effects of the second embodiment, the third embodiment can similarly obtain the advantageous effects relating to the progress degree.

### Fourth Embodiment

Next, an ultrasonic diagnosis apparatus according to a fourth embodiment will be described with reference to FIG. 1.

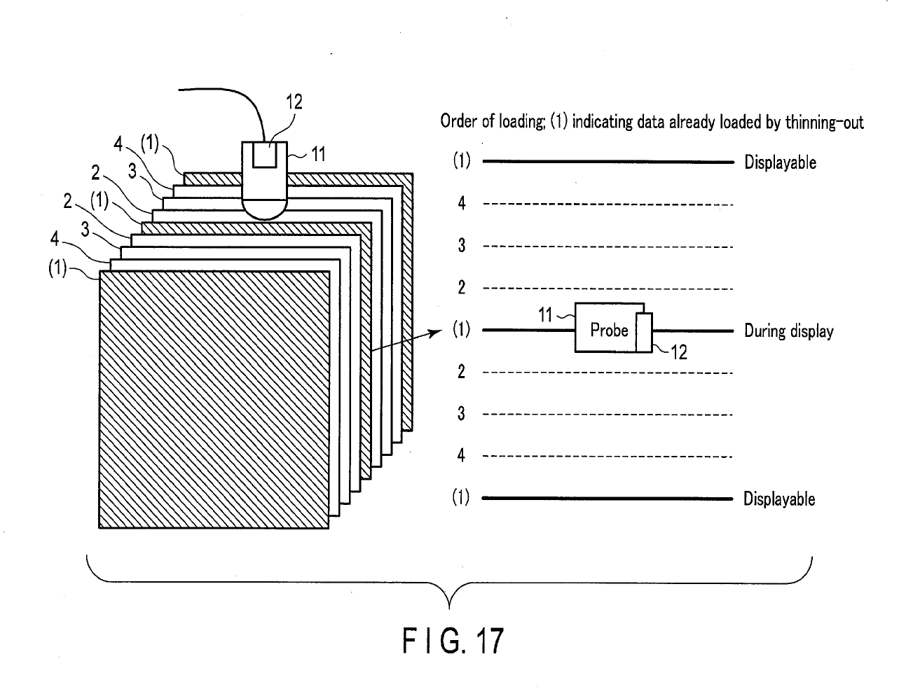

The fourth embodiment is a modification of the first embodiment, and has a configuration relating to a read-out position, as illustrated in FIG. 17. In an example of FIG. 17, image data, where were already loaded in the first load, are indicated by reference numeral (1), and image data, which are to be loaded in the second, third and fourth loads, are indicated by reference numerals 2, 3 and 4. Specifically, FIG. 17 illustrates that image data 2, 3 and 4 are loaded in the order from a position closest to the image data (1) which is currently displayed based on the output of the ultrasonic probe 11.

Accordingly, the background load process of the processing circuitry 23 is a process of loading residual data such that priority is given to the data at a read-out position close to the read-out position of the data corresponding to the position information which was acquired by the position acquisition circuitry 15 from the output of the position sensor 12.

A supplemental description is given. After the completion of registration, the fusion transitions to a synchronous state. Then, based on the position and angle in the position information, a position at which the scan plane of the ultrasonic image and the volume intersect is calculated, and image data is successively read out from the read-out position of image data close to the position of intersection.

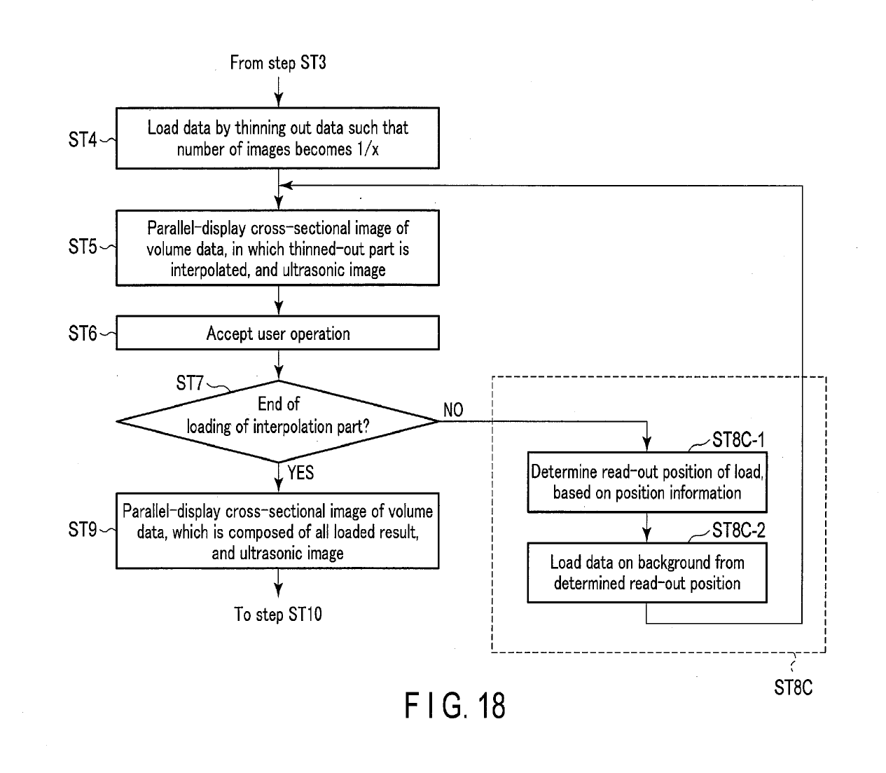

Next, the operation of the ultrasonic diagnosis apparatus with the above-described configuration will be described with reference to a flowchart of FIG. 18.

Now, steps ST1 to ST7 are executed in the same manner as described above. In step ST7, if it is determined that the loading of the interpolation part is not to be ended, the ultrasonic diagnosis apparatus 10 executes, in place of the above-described step ST8, step ST8C which includes ST8C-1 and ST8C-2.

Specifically, the background load process of the processing circuitry 23 determines the read-out position of residual data such that priority is given to the data at a read-out position close to the read-out position of the data corresponding to the position information which was acquired by the position acquisition circuitry 15 (step ST8C-1).

Subsequently, the background load process of the processing circuitry 23 transmits a load request including the determined read-out position and read-out intervals to the server apparatus 30 on the background. Thereby, the processing circuitry 23 loads residual data from the determined read-out position on the background (step ST8C-2), and goes to step ST5 of the second and following times.

Subsequently, the process of step ST5 onwards is executed in the same manner as described above.

As described above, according to the present embodiment, the residual data is loaded such that priority is given to the data at a read-out position close to the read-out position of the data corresponding to the acquired position information. By this configuration, in addition to the advantageous effects of the first embodiment, the image quality can be improved in the order from data close to the currently display slice image. Thus, compared to the case of improving the image quality on average over the entire range of volume data, the image quality of a necessary range can quickly be improved. For example, if a target is searched and alignment is executed, the density of image data can be increased with priority to a part near the position of the ultrasonic image that is to be registered.

In the meantime, although the fourth embodiment was described as being implemented as the modification of the first embodiment, the restriction to this is unnecessary, and the fourth embodiment may be implemented as a modification of the second or third embodiment. Thereby, in addition to the advantageous effects of the second or third embodiment, the fourth embodiment can similarly obtain the advantageous effects relating to the read-out position.

### Fifth Embodiment

Next, an ultrasonic diagnosis apparatus according to a fifth embodiment will be described with reference to FIG. 1.

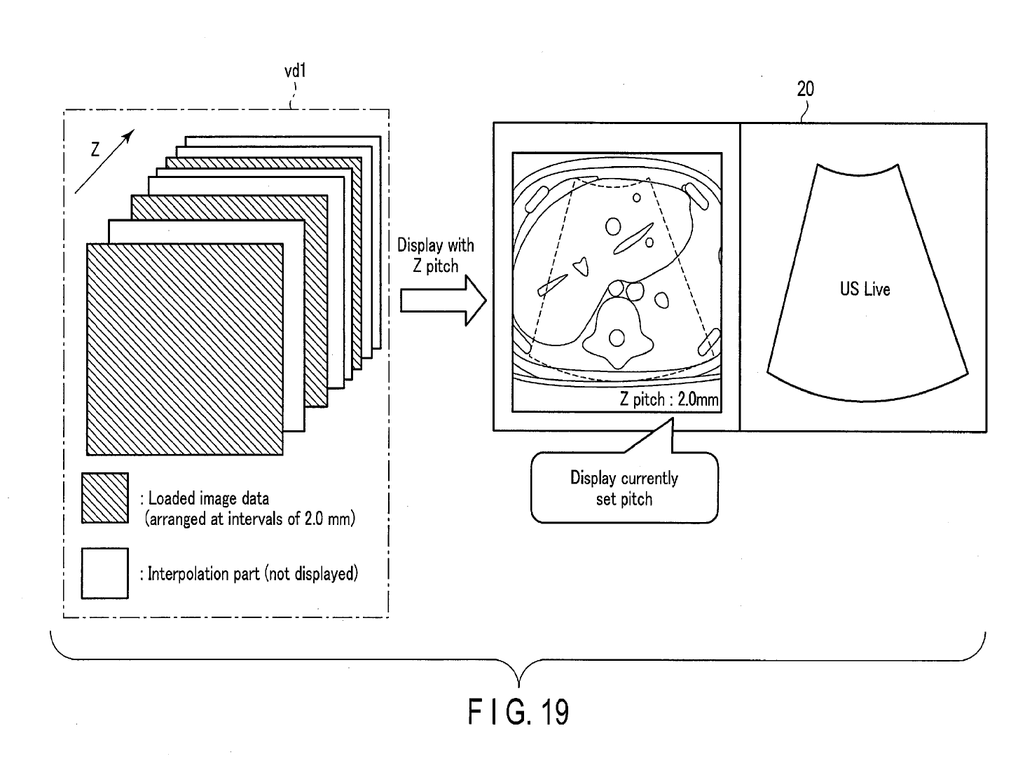

The fifth embodiment is a modification of the first embodiment, and has a configuration relating to the pitch of image feed, as illustrated in FIG. 19. In an example of FIG. 19, loaded image data are disposed at intervals of 2 mm in a line-of-sight direction (Z direction) which is perpendicular to slice images, and the slice images are displayed at equal intervals. Specifically, FIG. 19 illustrates that, while slice images corresponding to the loaded image data are displayed, slice images corresponding to the interpolation part are not displayed.

Accordingly, the control process by the control program 23d of the processing circuitry 23 includes a process of setting the pitch of slice image feed at a time of interlock-displaying slice images in the line-of-sight direction perpendicular to the slice images, and controlling the display circuitry 20 so as to further display the set pitch.

A supplemental description is given. While the slice image (MPR image) is being displayed, the pitch is set at a fixed interval when the display cross section is fed in the line-of-sight direction, and the set pitch is displayed on the GUI. Here, the pitch is basically set by the user, but the pitch may be automatically set in accordance with the reconstruction method of volume data. The interval of 2 mm is merely an example, and the interval may be changed to other values.

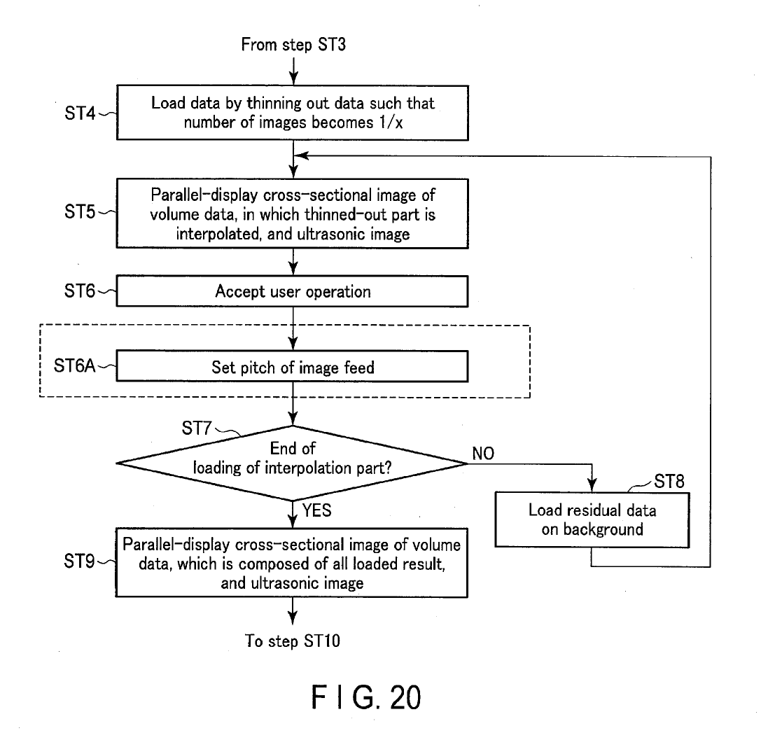

Next, the operation of the ultrasonic diagnosis apparatus with the above-described configuration will be described with reference to a flowchart of FIG. 20.

Now, steps ST1 to ST6 are executed in the same manner as described above. In the user operation acceptance state of step ST6, in the same manner as described above, the ultrasonic image and the slice image of volume data, which are registered, are interlock-displayed. Here, it is assumed that in the ultrasonic diagnosis apparatus 10, the pitch of slice image feed at the time of interlock-displaying slice images in the line-of-sight direction perpendicular to the slice images was input by the user operation. The processing circuitry 23 sets the input pitch of slice image feed (step ST6A), and controls the display circuitry 20 so as to further display the set pitch.

Subsequently, the process of step ST7 onwards is executed in the same manner as described above.

As described above, according to the present embodiment, the pitch of slice image feed at the time of interlock-displaying slice images in the line-of-sight direction perpendicular to the slice images is set, and the display circuitry 20 is controlled so as to further display the set pitch. Accordingly, in addition to the advantageous effects of the first embodiment, the pitch of slice image feed can be presented to the user.

A supplemental description will be given. According the present embodiment, the pitch of slice image feed is set at the read-out interval at the time of loading. Thereby, while slice images corresponding to the loaded image data are displayed, slice images corresponding to the interpolation part are not displayed. Therefore, even at the stage of loading which is being executed, the image quality of the slice image, which is displayed, can be kept at high quality.

In the meantime, although the fifth embodiment was described as being implemented as the modification of the first embodiment, the restriction to this is unnecessary, and the fifth embodiment may be implemented as a modification of each of the second to fourth embodiments. Thereby, in addition to the advantageous effects of the second to fourth embodiments, the fifth embodiment can similarly obtain the advantageous effects relating to the pitch of image feed.

### Sixth Embodiment

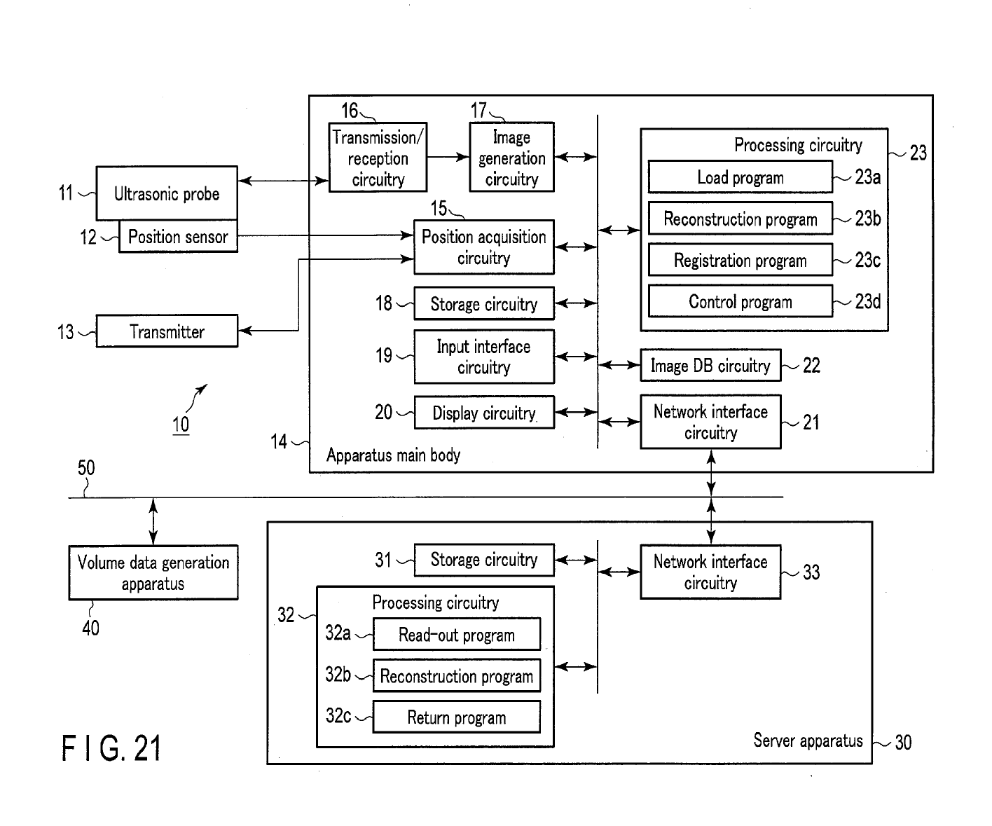

FIG. 21 is a schematic view illustrating an ultrasonic diagnosis apparatus and a server apparatus according to a sixth embodiment, and a peripheral configuration thereof.

The sixth embodiment is a modification of the first embodiment. As illustrated in FIG. 21, compared to the configuration illustrated in FIG. 1, the processing circuitry 32 of the server apparatus 30 executes a read-out process by a read-out program 32a, like the above-described read-out function, and executes a reconstruction process by a reconstruction program 32b and a return process by a second return program 32c, in place of the first return function.

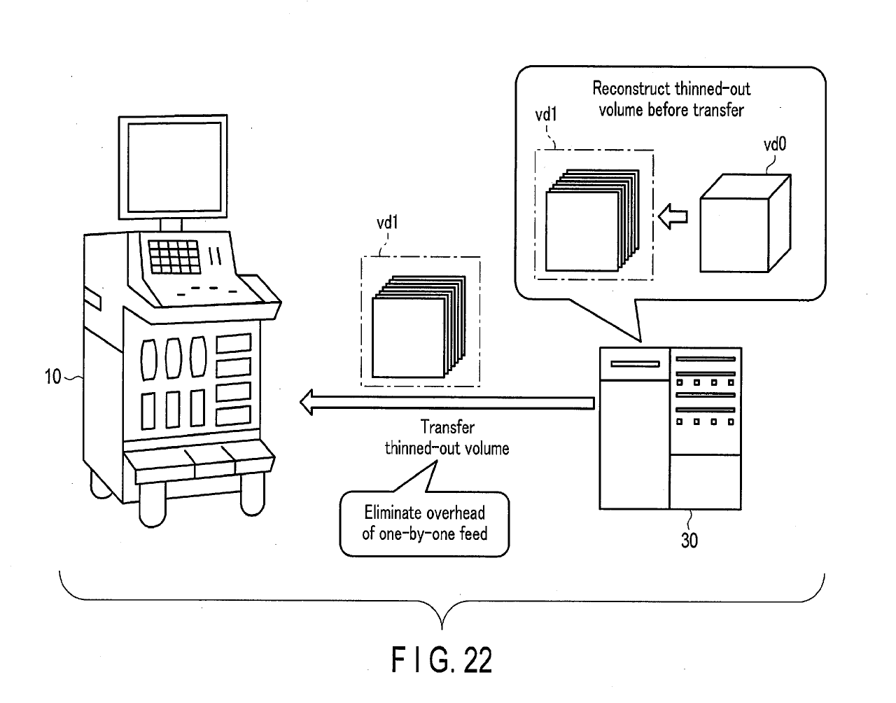

Here, the reconstruction process by the reconstruction program 32b is a process of reconstructing volume data of a smaller data amount than the volume data in the storage circuitry 31, from the read-out partial data. The return process by the second return program 32c is a process of returning (transferring) the reconstructed volume data to the transmission source of the load request. For example, as illustrated in FIG. 22, the processing circuitry 32 reconstructs, before transfer (return), volume data vd1 which is obtained by thinning out prestored volume data vd0, and transfers the reconstructed volume data vd1 to the ultrasonic diagnosis apparatus. Thereby, an overhead of transferring image data one by one can be eliminated.

The ultrasonic diagnosis apparatus 10 can interlock-display the slice image based on the volume data, which was loaded by transmitting the load request to the server apparatus 30, and the ultrasonic image based on the output of the ultrasonic probe, in accordance with the position of the ultrasonic probe 11.

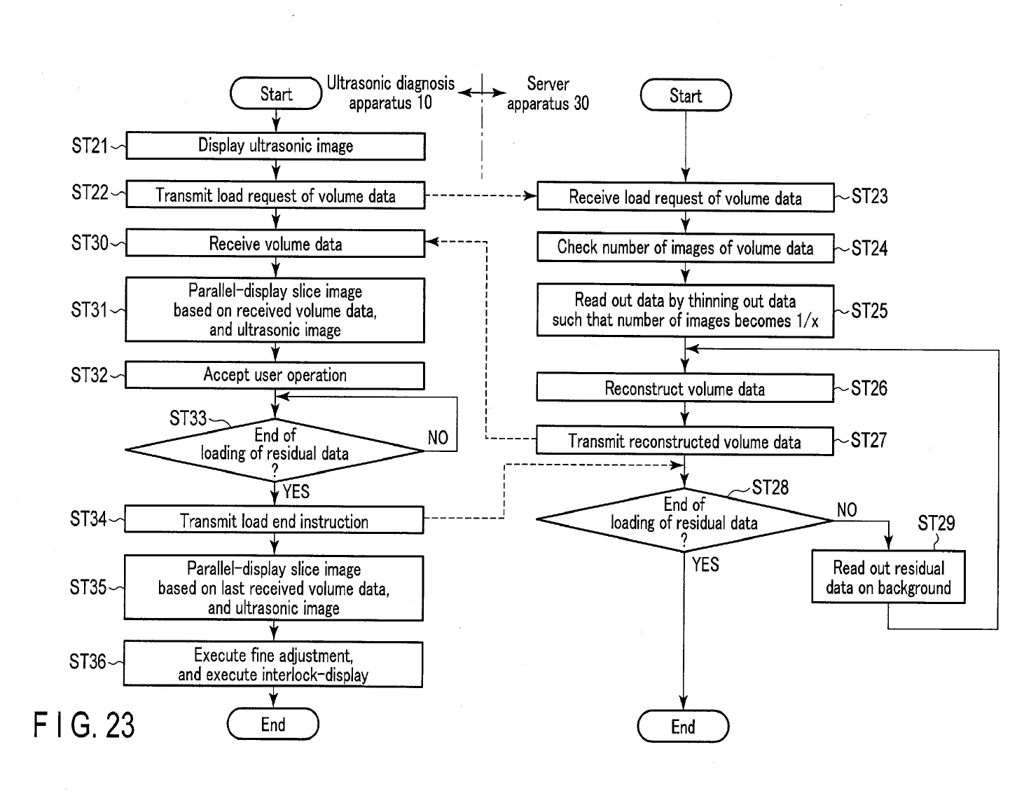

Next, the operation of the server apparatus and ultrasonic diagnosis apparatus having the above-described configurations will be described with reference to a flowchart of FIG. 23.

In the ultrasonic diagnosis apparatus 10, by an operation of the user such as a doctor, the ultrasonic probe 11 is put in contact with a subject, the inside of the subject is scanned by an ultrasonic beam, and an ultrasonic image is generated based on an output of the ultrasonic probe 11. Thereby, in the ultrasonic diagnosis apparatus 10, the ultrasonic image is displayed on the display circuitry 20 (step ST21).

In the processing circuitry 23 of the ultrasonic diagnosis apparatus, for example, the server apparatus 30 is designated as a load source by the user's operation, and volume data of a load target is designated. Then, a load request including the designation of the volume data is transmitted to the server apparatus 30 (step ST22).

Upon receiving the load request (step ST23), the processing circuitry 32 of the server apparatus 30 reads out the volume data, which is designated in the load request, from the storage circuitry 31, and checks the number of images of the volume data (step ST24).

Subsequently, the processing circuitry 32 determines the read-out position and read-out interval by thinning out the data such that the number of images becomes 1/x, and reads out predetermined data from the volume data in the storage circuitry 31, based on the read-out position and read-out interval (step ST25). The predetermined data is, for example, partial data of the volume data. Specifically, based on the read-out position and read-out interval, the processing circuitry 32 reads out reference image data serving as a reference, from the read-out position in the volume data, and writes identification information of the read-out image data into the image arrangement map, based on the read-out position of the read-out image data. In addition, the processing circuitry 32 reads out image data in the storage circuitry 31 from the reference image data at read-out intervals, and writes the identification information of the image data into the image arrangement map in accordance with the read-out position of the image data.

These steps ST23 to ST25 are an example of the read-out process which is realized by the processing circuitry 32 reading out the read-out program 32a from the storage circuitry 31 and executing the read-out program 32a. The read-out process is a process in which the processing circuitry 32 reads out predetermined data from the volume data in the storage circuitry 31, based on the load request transmitted from the ultrasonic diagnosis apparatus 10.

Next, the processing circuitry 32 reconstructs volume data exhibiting the thinned-out state in step ST25, based on each read-out image data (step ST26). Specifically, the processing circuitry 32 reconstructs volume data of three-dimensional images in which slice images are arranged at read-out intervals, without interpolating the thinned-out part (the part of 1−1/x). This step ST26 is an example of the reconstruction process which is realized by the processing circuitry 32 reading out the reconstruction program 32b from the storage circuitry 31 and executing the reconstruction program 32b. This reconstruction process is a process of reconstructing volume data of a smaller data amount than the volume data in the storage circuitry 31, from the partial data which was read out by the read-out process.

Thereafter, the processing circuitry 32 transmits the volume data exhibiting the thinned-out state and the image arrangement map to the ultrasonic diagnosis apparatus 10 (step ST27). This step ST27 is an example of the return process which is realized by the processing circuitry 32 reading out the return program 32c from the storage circuitry 31 and executing the return program 32c. This return process is a process of returning the reconstructed volume data, which was reconstructed by the reconstruction process, to the transmission source of the load request.

After step ST27, the processing circuitry 32 determines whether the loading of the residual part is to be ended or not (step ST28). If “NO” in step ST28, the processing circuitry 32 reads out the residual data on the background (step ST29), and goes to step ST26. For example, if a load end instruction is not received and there is unloaded residual data, the processing circuitry 32 determines “NO” in step ST28. Here, the load end instruction is used for stopping further loading, in the same manner as described above.

On the other hand, the processing circuitry 23 of the ultrasonic diagnosis apparatus 10 receives the volume data and image arrangement map, which were transmitted from the server apparatus 30 (step ST30), and writes the volume data and image arrangement map into the storage circuitry 18.

After the completion of reception, the processing circuitry 23 interpolates and reconstructs the volume data in the storage circuitry 18, based on the image arrangement map, and the processing circuitry 23 parallel-displays the slice image, which is based on the reconstructed volume data, and the ultrasonic image on the display circuitry 20 (step ST31). In step ST30 of the second and following times, the processing circuitry 23 reconstructs new volume data by replacing the interpolation part in the previously reconstructed volume data with image data in the newly received volume data. Thereafter, the processing circuitry 23 updates the current volume data with the new volume data, and parallel-displays the slice image of the updated volume data and the ultrasonic image.

After the start of the parallel-display in step ST31, the processing circuitry 23 transitions to the user operation acceptance state for accepting a user operation (step ST32). In the user operation acceptance state, registration of the position/angle information of the slice image of the volume data and the ultrasonic image is executed by the user's operation.

After the completion of registration, the ultrasonic diagnosis apparatus 10 interlock-displays the ultrasonic image and the slice image of the volume data in accordance with the position of the ultrasonic probe 11.

Thereafter, in the user operation acceptance state, while the user visually recognizes the ultrasonic image and slice image of the volume data which are interlock-displayed, the user applies puncture or RFA treatment to the subject.

On the other hand, in the user operation acceptance state of step ST32, while the processing circuitry 23 accepts a user operation, the processing circuitry 23 determines whether the loading of the residual part is to be ended or not (step ST33). For example, if a load end instruction is not input and there is unloaded residual data, the processing circuitry 23 determines “NO”, and continues the determination of step ST33.

In the determination of step ST33, for example, if the load end instruction is input by the user operation or if the loading of all data is completed, the processing circuitry 23 determines that the loading is to be ended, and transmits the load end instruction to the server apparatus 30 (step ST34).

After step ST34, the processing circuitry 23 reconstructs new volume data by replacing the interpolation part in the previously reconstructed volume data with image data in the last received volume data. Thereafter, the processing circuitry 23 updates the current volume data with the new volume data, and parallel-displays the slice image of the updated volume data and the ultrasonic image on the display circuitry 20 (step ST35). During this parallel-display, the processing circuitry 23 finely adjusts the alignment result and registration result between the slice image of the volume data and the ultrasonic image in accordance with the user operation, and executes interlock-display (step ST36). Thereafter, in the same manner as described above, while the user visually recognizes the ultrasonic image and slice image of the volume data which are interlock-displayed, the user applies puncture or RFA treatment to the subject.

As has been described above, according to the present embodiment, the server apparatus 30 reads out predetermined data from the volume data, and reconstructs volume data of a smaller data amount than the volume data in the storage circuitry 31, from the read-out data. Thereafter, the reconstructed volume data is returned to the transmission source of the load request.

Thereby, in addition to the advantageous effects of the first embodiment, the overhead of the process, in which the server apparatus 30 transmits the image data one by one, can be eliminated.

A supplemental description is given. The server apparatus 30 does not transmit the image one by one, but transmits the volume data of a small data amount. Therefore, the overhead of the transmission process can be eliminated. Here, the volume data of the small data amount may be created by the above-described thinning-out process, or may be created from the image data at a position near the currently displayed position. Besides, the server apparatus 30 may transmit the volume data on the background until receiving the load end instruction from the ultrasonic diagnosis apparatus 10, as described above, or may transmit the volume data at each time by receiving the load request from the ultrasonic diagnosis apparatus 10.

According to at least one of the above-described embodiments, predetermined data is loaded from prestored volume data, and volume data is reconstructed from the loaded data. In addition, the slice image of the reconstructed volume data and the ultrasonic image based on the output of the ultrasonic probe are displayed in parallel.

Thus, according to the embodiments, the load time of volume data can be shortened, compared to the case of loading all data in the volume data. Therefore, a hindrance to the workflow due to the load time of volume data can be reduced.

The term “processor” used in the above description means, for example, a CPU (Central Processing Unit), a GPU (Graphics Processing Unit), or circuitry such as an ASIC (Application Specific Integrated Circuit), or a programmable logic device (e.g. SPLD (Simple Programmable Logic Device), CLPD (Complex Programmable Logic Device), FPGA (Field Programmable Gate Array)). The processor realizes functions by reading out and executing programs stored in the memory circuitry. In the meantime, instead of storing programs in the memory circuitry, such a configuration may be adopted that programs are directly incorporated in the circuitry in the processor. In this case, the processor realizes functions by reading out and executing programs stored in the circuitry. Each of the processors in the embodiments may not be configured as single circuitry for each processor. A plurality of independent circuitries may be constructed as a single processor, and the functions of the processor may be realized. Furthermore, a plurality of structural elements in FIG. 1, FIG. 12 and FIG. 21 may be integrated in a single processor, and the functions of the processor may be realized.

The position acquisition circuitry 15 in the first embodiment is an example of position acquisition circuitry in the claims. The load program 23a, reconstruction program 23b, registration program 23c and control program 23d in the first embodiment are examples of a load process, a reconstruction process, a registration process and a control process which processing circuitry in the claims executes. The display circuitry 20 in the first embodiment is an example of a display in the claims. The background load process and update process in the first embodiment are examples of a background load process and an update process in the claims. The storage circuitry 31, read-out program 32a, reconstruction program 32b and second return program 32c in the sixth embodiment are examples of storage circuitry, and a read-out process, a reconstruction process and a return process which the processing circuitry executes, which are recited in the claims.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. An ultrasonic diagnosis apparatus comprising:
position acquisition circuitry configured to acquire position information of an ultrasonic probe;
processing circuitry configured to execute a load process of loading predetermined data from volume data stored in other apparatus, and to execute a reconstruction process of reconstructing volume data from the loaded data; and
a display configured to display in parallel a slice image of the reconstructed volume data and an ultrasonic image based on an output of the ultrasonic probe,
wherein the processing circuitry is configured to execute a registration process in such a manner as to register a position of the ultrasonic image based on the output of the ultrasonic probe and a position of the slice image based on the loaded data, and to execute a control process of controlling, after the registration process, the display in such a manner as to interlock-display the ultrasonic image and the slice image in accordance with the position information.

2. The ultrasonic diagnosis apparatus of claim 1, wherein the predetermined data is thinned-out data, data bundled in units of a predetermined number of data, data at a predetermined imaging time, or data of an extracted predetermined region.

3. The ultrasonic diagnosis apparatus of claim 1, wherein the registration process includes a process of accepting an operation for registration.

4. The ultrasonic diagnosis apparatus of claim 1, wherein the registration process includes a process of executing automatic registration, based on position information which the ultrasonic image and the slice image have, or based on the ultrasonic image and the slice image.

5. The ultrasonic diagnosis apparatus of claim 1, wherein the load process includes a background load process of loading data, which has not been loaded by the loading, on a background, while the ultrasonic probe is being operated, and
the reconstruction process includes an update process of updating volume data for displaying the slice image, by the data loaded on the background and partial data which has been loaded by the loading.

6. The ultrasonic diagnosis apparatus of claim 5, wherein the background load process includes a process of loading the data which has not been loaded by the loading, such that priority is given to data at a read-out position close to a read-out position of data corresponding to the acquired position information.

7. The ultrasonic diagnosis apparatus of claim 1, wherein the volume data is composed of a plurality of image data, and
the load process includes a process of loading, as the predetermined data, partial image data which is obtained by thinning out the plurality of image data.

8. The ultrasonic diagnosis apparatus of claim 5, wherein the control process includes a process of calculating a progress degree of loading by the background load process, and controlling, after the updating by the update process, the display in such a manner to further display the calculated progress degree.

9. The ultrasonic diagnosis apparatus of claim 1, wherein the control process includes a process of setting a pitch of slice image feed at a time of executing the interlock-displaying of the slice image in a line-of-sight direction perpendicular to the slice image, and controlling the display in such a manner as to further display the set pitch.

10. A non-transitory computer-readable storage medium having stored thereon a program which is executable by processor of a server apparatus which is communicable with an ultrasonic diagnosis apparatus that is capable of interlock-displaying a slice image based on volume data, which was loaded by transmitting a load request, and an ultrasonic image based on an output of an ultrasonic probe, in accordance with a position of the ultrasonic probe, the server apparatus including storage circuitry prestoring volume data, the program comprising:
a first program code of causing the processor to execute a process of reading out predetermined data from the volume data in the storage circuitry, based on the load request transmitted from the ultrasonic diagnosis apparatus;
a second program code of causing the processor to execute a process of reconstructing, from the read-out data, volume data of a smaller data amount than the volume data in the storage circuitry; and
a third program code of causing the processor to execute a process of returning the reconstructed volume data to a transmission source of the load request.
